(19) United States
(12) Patent Application Publication
(10) Pub. No.: US 2023/0414374 A1
(43) Pub. Date: Dec. 28, 2023

(54) Interbody spacer with non-screw anchor

(71) Applicant: Joey Reglos, Dumaguete City (PH)

(72) Inventor: Joey Reglos, Dumaguete City (PH)

(21) Appl. No.: 18/213,878

(22) Filed: Jun. 25, 2023

Related U.S. Application Data
(60) Provisional application No. 63/355,533, filed on Jun. 24, 2022.

(51) Int. Cl. A61F 2/44; A61F 2/30

(52) U.S. Cl. CPC A61F 2/442; A61F 2/30749; A61F 2002/30428; A61F 2002/30331; A61F 2002/30576; A61F 2002/30518; A61F 2002/30517; A61F 2002/30492

(57) ABSTRACT

An interbody spacer for the spine comprises an anchor assembly removably connectable to a cage. The anchor assembly includes at least two non-screw bone anchors. With insertion of the anchor assembly into the cage, at least one anchor slides against a ramp from an undeployed configuration to rotate up with respect to the plate and out of an upper exit opening of the cage for anchoring into an upper vertebral body and a second anchor slides against a ramp to rotate down with respect to the plate and out of a lower exit opening of the cage for anchoring into a lower vertebral body. The anchor assembly locks with respect to the cage by way of deflectable locking tabs.

### CROSS-REFERENCE TO RELATED APPLICATIONS

This application claims priority to and benefit of U.S. Provisional Patent Application Ser. No. 63/355,533 entitled “Cervical spacer with non-screw anchor” filed on Jun. 24, 2022 which is incorporated herein by reference in its entirety.

### FIELD OF THE INVENTION

This application relates generally to spinal implants, and in particular, interbody spacers for the spine and associated insertion instrument.

### BACKGROUND

Back pain can be caused by a variety of factors including but not limited to the rupture or degeneration of one or more intervertebral discs due to degenerative disc disease, spondylolisthesis, deformative disorders, trauma, tumors and the like. In such cases, pain typically results from compression or irritation of spinal nerve roots arising from reduced spacing between adjacent vertebrae, a damaged disc and or misalignment of the spine resulting from the injury or degeneration.

Common forms of treating such pain include various types of surgical procedures in which a damaged disc may be partially or totally excised. After the disc space is prepared, one or more implants are inserted between the adjacent vertebrae in an effort to restore the natural spacing and alignment between the vertebrae, so as to relieve the compression, irritation or pressure on the spinal nerve or nerves and, thereby, eliminate or significantly reduce the pain that the patient is experiencing. Typically, one or more implants are used together with substances that encourage bone ingrowth to facilitate fusion between adjacent vertebrae and achieve immobilization of adjacent bones. Surgeons insert these intervertebral devices to adjunctively facilitate bone fusion in between and into the contiguous involved vertebrae. This fusion creates a new solid bone mass and provides weight bearing support between adjacent vertebral bodies which acts to hold the spinal segment at an appropriate biomechanically restored height as well as to stop motion in a segment of the spine and alleviate pain.

In a typical procedure, the adjacent vertebrae must be distracted apart by a substantial amount in order to allow the surgeon to advance the implant with relatively little resistance along the delivery path. As the implant is being delivered along the delivery path, the surgeon aligns and positions the implant at the target location of implantation. If static spacers having a fixed height are employed, the right-sized spacer is selected from a plurality of spacers. Typically, bone screws are employed to anchor the implant. These screws must be delivered individually and screwed into the vertebrae. Typically, three screws are employed and the process of individual delivery of screws increases operative time and may cause shifting of the implant as the upper or lower side of the implant is stabilized before the other side.

Over time, the interface between the screws and the bone may present some problems of stability. Due to the anatomical structure of the spine and the extreme anatomical forces that are brought to bear on the skeleton and transmitted to the vertebral bodies, the screws securing the interbody spacer to the spine may vibrate or toggle out of position or otherwise, move with respect to the cage and become dislodged. As a result, bone screws securing the plate to the spine may move or back out of the vertebral body and implant. Loosened screws may result in instability of the joint and lead to increased pain for the patient. Implants for the cervical spine must be small and place further demands on stability and design.

Therefore, there is a need to provide a new and improved interbody spacer that prevents fasteners from backing out and also from being loosened with respect to the cage. Furthermore, there is a need for the implant to withstand anatomical forces and be easily and quickly implantable. This invention, as described herein, sets forth an improved interbody spacer that meets these needs and is well-adapted for constraints encountered in all regions of the spine.

### SUMMARY OF THE INVENTION

According to one aspect of the invention, an interbody spacer is provided. The interbody spacer comprises a cage including a top surface and a bottom surface interconnected by a left sidewall, a right sidewall and an endwall defining an interior having rear opening. The cage includes a left locking slot formed in the left sidewall and a right locking slot formed in the right sidewall. The cage includes at least one ramped surface in the interior interconnected with a corresponding exit opening in the top surface. The cage includes at least one ramped surface in the interior interconnected with a corresponding exit opening in the bottom surface. The cage includes a threaded bore in the interior facing the rear opening. The interbody spacer includes an anchor assembly. The anchor assembly includes a plate configured for insertion into the rear opening and into the interior. The plate has an instrument opening aligned with the threaded bore. The plate includes a left locking tab configured to flex with respect to the plate and enter into locking engagement with the left locking slot. The plate includes a right locking tab configured to flex with respect to the plate and enter into locking engagement with the right locking slot. The anchor assembly includes at least two non-screw bone anchors rotatably connected to the plate. The interbody spacer is configured such that insertion of the anchor assembly into the cage causes the bone anchors to rotate out of the top and bottom exit openings and the left and right locking tabs to lock into the left and right locking slots.

According to another aspect of the invention, an interbody spacer is provided. The interbody spacer includes a cage defining an interior and a rear opening. The interbody spacer includes an anchor assembly sized and configured for insertion into the rear opening and into the interior of the cage. The anchor assembly includes at least two non-screw bone anchors rotatably attached to an endplate.

According to another aspect of the invention, an interbody spacer system is provided. The interbody spacer system includes an interbody spacer. The interbody spacer includes a cage and an anchor assembly. The anchor assembly includes at least two non-screw bone anchors rotatably connected to a plate. The plate includes an instrument opening having a cross-sectional shape. The interbody spacer system includes an insertion instrument including a main body having a cross-sectional shape corresponding to the shape of the instrument opening and sized and configured for insertion into the instrument opening.

### BRIEF DESCRIPTION OF THE DRAWINGS

FIG. 1 is a top perspective view of an interbody spacer locked in a deployed configuration with a secondary lock according to the present invention.

FIGS. 2A-2I are various views of a cage of the interbody spacer of FIG. 1.

FIGS. 3A-3H are various views of an anchor assembly of the interbody spacer of FIG. 1.

FIGS. 4A-4B are views of a plate of the anchor assembly of FIGS. 3A-3H.

FIG. 5 is a side view of a bone anchor of the anchor assembly of FIGS. 3A-3H.

FIG. 6 is a top perspective view of a pin of the anchor assembly of FIGS. 3A-3H.

FIG. 7 is a top perspective view of an optional secondary lock of the interbody spacer of FIG. 1 according to the present invention.

FIGS. 8A-8C are various views of the anchor assembly of FIGS. 3A-3H disconnected from the cage of FIGS. 2A-2I in an anchor-undeployed, partially inserted, and unlocked position according to the present invention.

FIGS. 9A-9F are various views of the anchor assembly of FIGS. 3A-3H connected to the cage of FIGS. 2A-2I in an anchor fully deployed, fully inserted and locked position according to the present invention.

FIGS. 10A-10D are various views of the anchor assembly of FIGS. 3A-3H connected to the cage of FIGS. 2A-2I in an anchor-fully-deployed, fully inserted, locked position and the anchor assembly being also locked with the secondary lock of FIG. 7 according to the present invention.

FIGS. 11A-11I are various views of another variation of a cage for an interbody spacer according to the present invention.

FIGS. 12A-12H are various views of an anchor assembly for use with the cage of FIGS. 11A-11I according to the present invention.

FIGS. 13A-13B are views of a plate of the anchor assembly of FIGS. 12A-12H.

FIG. 14 a side view of a bone anchor of the anchor assembly of FIGS. 12A-12H.

FIG. 15 is a top perspective view of a pin of the anchor assembly of FIGS. 12A-12H.

FIG. 16 is a top perspective view of an optional secondary lock for use with the cage of FIGS. 11A-11I and with the anchor assembly of FIGS. 12A-12H according to the present invention.

FIGS. 17A-17C are various views of the anchor assembly of FIGS. 12A-12H disconnected from the cage of FIGS. 11A-11I in an anchor-undeployed, partially inserted, and unlocked position according to the present invention.

FIGS. 18A-18F are various views of the anchor assembly of FIGS. 12A-12H connected to the cage of FIGS. 11A-11I in an anchor fully deployed, fully inserted and locked position according to the present invention.

FIGS. 19A-19D are various views of the anchor assembly of FIGS. 12A-12H connected to the cage of FIGS. 11A-11I in an anchor-fully-deployed, fully inserted, locked position and further the anchor assembly being locked with the secondary lock of FIG. 16 according to the present invention.

FIGS. 20A-20I are various views of an inserter according to the present invention.

FIGS. 21A-21K are various views of the inserter of FIGS. 20A-20I connected to the anchor assembly of FIGS. 12A-12H or connected to the anchor assembly of FIGS. 12A-12H and cage of FIGS. 11A-11I depicting various stages of deployment of the interbody spacer with the inserter according to the present invention.

FIG. 22A is a top perspective view of the inserter of FIGS. 20A-20I connected to the interbody spacer outside of a spinal segment in an undeployed configuration according to the present invention.

FIG. 22B is a top perspective view of the inserter of FIGS. 20A-20I connected to the interbody spacer with the interbody spacer located between two vertebral bodies in an undeployed configuration according to the present invention.

FIG. 22C is a top perspective view of the inserter of FIGS. 20A-20I connected to the interbody spacer with the interbody spacer located between two vertebral bodies in a deployed configuration according to the present invention.

FIG. 22D is a top perspective view of a bone graft funnel connected to the interbody spacer without the secondary lock and bone graft tamp according to the present invention.

FIG. 23A is a top perspective rear view of an interbody spacer locked in a deployed configuration with a secondary lock according to the present invention.

FIG. 23B is a top perspective front view of an interbody spacer locked in a deployed configuration with a secondary lock according to the present invention.

FIG. 23C is a top view of an interbody spacer locked in a deployed configuration with a secondary lock according to the present invention.

FIG. 23D is a cross-sectional view of an interbody spacer taken along line P-P of FIG. 23C according to the present invention.

FIG. 23E is a side elevational view of an interbody spacer according to the present invention.

FIG. 24A is a top perspective rear view of a cage of the interbody spacer of FIG. 23 according to the present invention.

FIG. 24B is a top perspective front view of a cage of the interbody spacer of FIG. 23 according to the present invention.

FIG. 24C is a side elevational view of a cage of the interbody spacer of FIG. 23 according to the present invention.

FIG. 24D is a rear elevational view of a cage of the interbody spacer of FIG. 23 according to the present invention.

FIG. 24E is a front elevational view of a cage of the interbody spacer of FIG. 23 according to the present invention.

FIG. 24F is a top view of a cage of the interbody spacer of FIG. 23 according to the present invention.

FIG. 24G is a cross-sectional view taken along line AA-AA of FIG. 24F of a cage according to the present invention.

FIG. 24H is a cross-sectional view taken along line AB-AB of FIG. 24F of a cage according to the present invention.

FIG. 24I is a cross-sectional view taken along line AC-AC of FIG. 24F of a cage according to the present invention.

FIG. 25A is top perspective rear view of an anchor assembly of the interbody spacer of FIG. 23 according to the present invention.

FIG. 25B is a top perspective front view of an anchor assembly of the interbody spacer of FIG. 23 according to the present invention.

FIG. 25C is a side elevational view of an anchor assembly of the interbody spacer of FIG. 23 according to the present invention.

FIG. 25D is a rear elevational view of an anchor assembly of the interbody spacer of FIG. 23 according to the present invention.

FIG. 25E is a front elevational view of an anchor assembly of the interbody spacer of FIG. 23 according to the present invention.

FIG. 25F is top perspective front, exploded view of an anchor assembly of the interbody spacer of FIG. 23 according to the present invention.

FIG. 26A is a top perspective rear view of a plate of an anchor assembly of the interbody spacer of FIG. 23 according to the present invention.

FIG. 26B is a top view of a plate of an anchor assembly of the interbody spacer of FIG. 23 according to the present invention.

FIG. 27 is a top view of a pin of the anchor assembly of the interbody spacer of FIG. 23 according to the present invention.

FIG. 28 is a top perspective view of a secondary lock of the interbody spacer of FIG. 23 according to the present invention.

FIG. 29A is a side elevational view of a bone anchor according to the present invention.

FIG. 29B is a bottom view of a bone anchor according to the present invention.

FIG. 29C is a top view of a bone anchor according to the present invention.

FIG. 29D is a top perspective view of a bone anchor according to the present invention.

FIG. 30A is a top perspective view of an anchor assembly in an undeployed configuration partially inserted into a cage according to the present invention.

FIG. 30B is a top view of an anchor assembly in an undeployed configuration partially inserted into a cage according to the present invention.

FIG. 30C is a cross-sectional view taken along line L-L of FIG. 30B of an anchor assembly in an undeployed configuration partially inserted into a cage according to the present invention.

FIG. 31A is a top perspective view of an anchor assembly in a fully inserted and deployed configuration of an interbody spacer according to the present invention.

FIG. 31B is a side elevational view of an interbody spacer with a fully inserted and deployed anchor assembly according to the present invention.

FIG. 31C is a top view of an interbody spacer with a fully inserted and deployed anchor assembly according to the present invention.

FIG. 31D is a cross-sectional view taken along line M-M of FIG. 31C of an interbody spacer with a fully inserted and deployed anchor assembly according to the present invention.

FIG. 31E is a top perspective rear view of an interbody spacer with four bone anchors according to the present invention.

FIG. 31F is a front elevational view of an interbody spacer with four bone anchors according to the present invention.

FIG. 31G is a top perspective view of a bone anchor assembly configured with four bone anchors according to the present invention.

FIG. 32A is a top view of an interbody spacer with a fully inserted and deployed anchor assembly with a secondary lock according to the present invention.

FIG. 32B is a cross-sectional view taken along line P-P of FIG. 32A of an interbody spacer with a fully inserted and deployed anchor assembly with a secondary lock according to the present invention.

FIG. 33 is a top perspective view of an inserter instrument and interbody spacer according to the present invention.

FIG. 34A is a top perspective sectional view of a distal end of an inserter instrument and anchor assembly according to the present invention.

FIG. 34B is a top perspective sectional view of a distal end of an inserter instrument and an anchor assembly according to the present invention.

FIG. 35A is a top perspective sectional view of a distal end of an inserter instrument connected to an anchor assembly and positioned before a cage according to the present invention.

FIG. 35B is a top perspective sectional view of a distal end of an inserter instrument connected to an anchor assembly and positioned before a cage according to the present invention.

FIG. 36A-36B is a top perspective sectional view of a distal end of an inserter instrument connected to an anchor assembly and partially inserted into a cage according to the present invention.

FIG. 37A is an interbody spacer configured for anterior lumbar interbody fusion according to the present invention.

FIG. 37B is the interbody spacer configured for anterior lumbar interbody fusion (ALIF) of FIG. 37A shown positioned within a disc space of a spinal column according to the present invention.

FIG. 38A is an interbody spacer configured for direct lateral interbody fusion (DLIF) according to the present invention.

FIG. 38B is the interbody spacer configured for direct lateral interbody fusion (DLIF) of FIG. 38A shown positioned within a disc space of a spinal column according to the present invention.

### DETAILED DESCRIPTION OF THE INVENTION

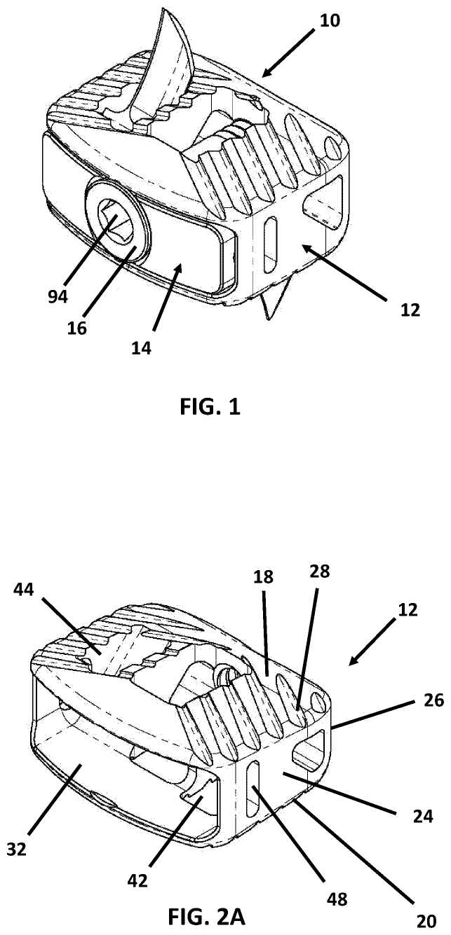

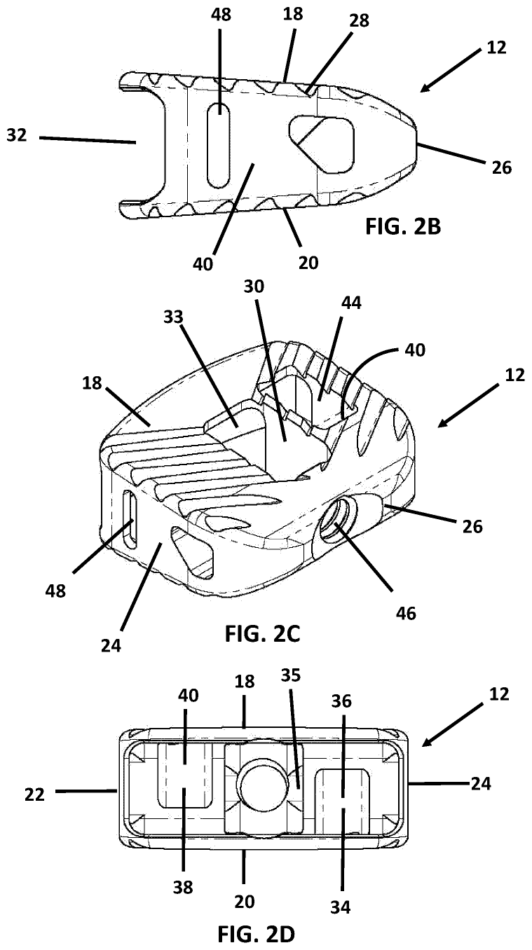

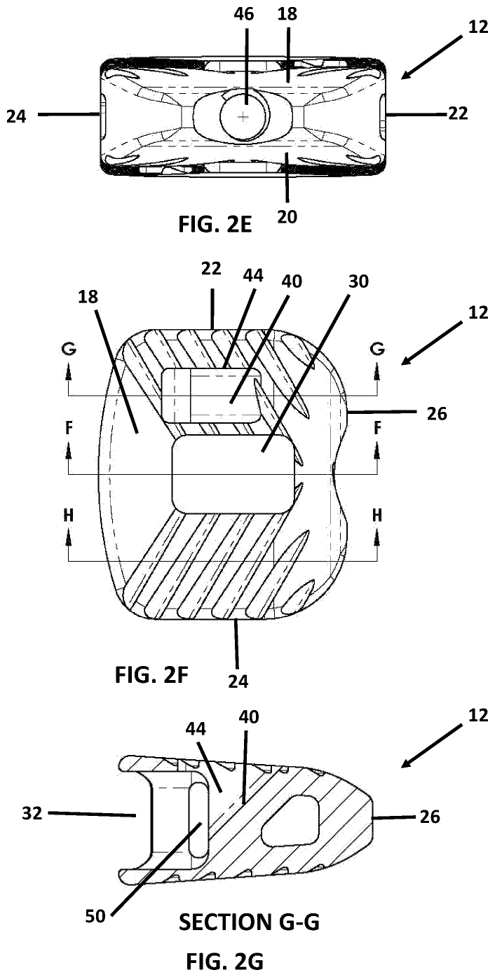

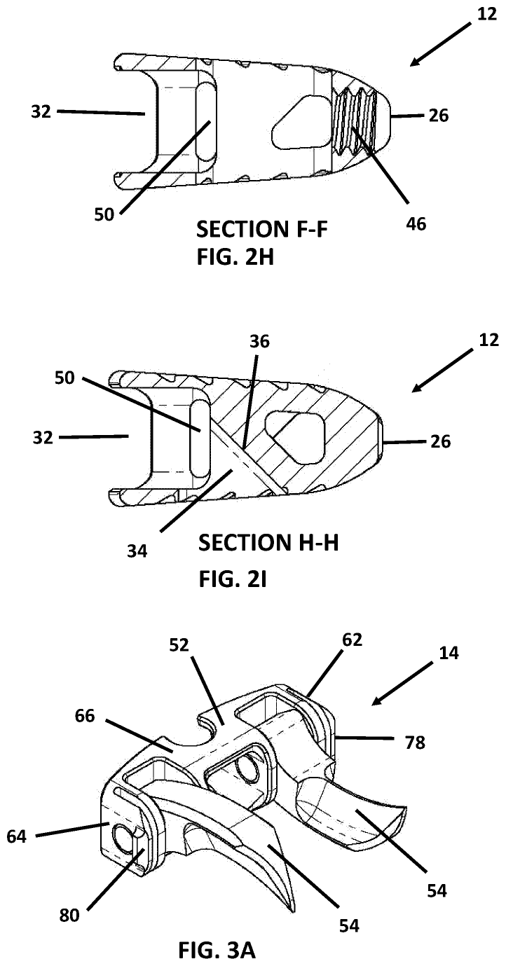

The present invention describes an interbody spacer 10 comprising a cage 12 removably connectable to an anchor assembly 14 with an optional secondary lock 16 as shown in FIG. 1. The cage 12 by itself is shown in FIGS. 2A-2I. The cage 12 includes a top endplate 18 and a bottom endplate 20 interconnected by a left sidewall 22, a right sidewall 24, and a leading end wall 26. The spacer 10 does not have a trailing end wall. Instead, the trailing end of the spacer 10 is provided with a rectangular-shaped rear opening 32 that extends between the top endplate and bottom endplate and between the left sidewall and right sidewall. The top endplate and bottom endplate each have ridges 28 for increasing traction against two adjacent vertebral bodies between which the spacer 10 is inserted. The cage 12 includes a central opening 30 extending vertically through the cage 12 between the top endplate and the bottom endplate. The central opening decreases the weight of the spacer and provides a location for bone graft placement for bone ingrowth to occur to further stabilize the spacer 10 with respect to the spine. The rear opening is interconnected with the central opening via a rectangular window 33. Along the same central horizontal longitudinal axis of the spacer, a threaded opening 46 is provided which opens at the leading end. The central opening is interconnected with the threaded opening 46 via a second or distal rectangular window 35. The rectangular windows help orientate a rectangular-shaped sleeve of an inserter instrument. Bone graft may be inserted through the rear opening, first/proximal rectangular window and delivered into the central opening after implantation of the spacer in the vertebral space or prior to implantation. The cage further includes a right anchor chamber 34 having a right ramped surface 36 and a left anchor chamber 38 having a left ramp surface 40. The cage includes a right chamber exit opening 42 formed in the bottom endplate and a left chamber exit opening 44 formed in the top endplate. The right exit opening 42 is rectangular in shape and defines the floor of the right chamber being in communication therewith. The left exit opening 44 is rectangular in shape and is defined in the ceiling of the left chamber. The right sidewall defines a vertically extending locking slot 48 and the left sidewall defines a vertically extending locking slot 50. The right ramp surface is angled downwardly to meet the front end of the right exit opening. The left ramp surface is angled upwardly to meet the front end of the left exit opening.

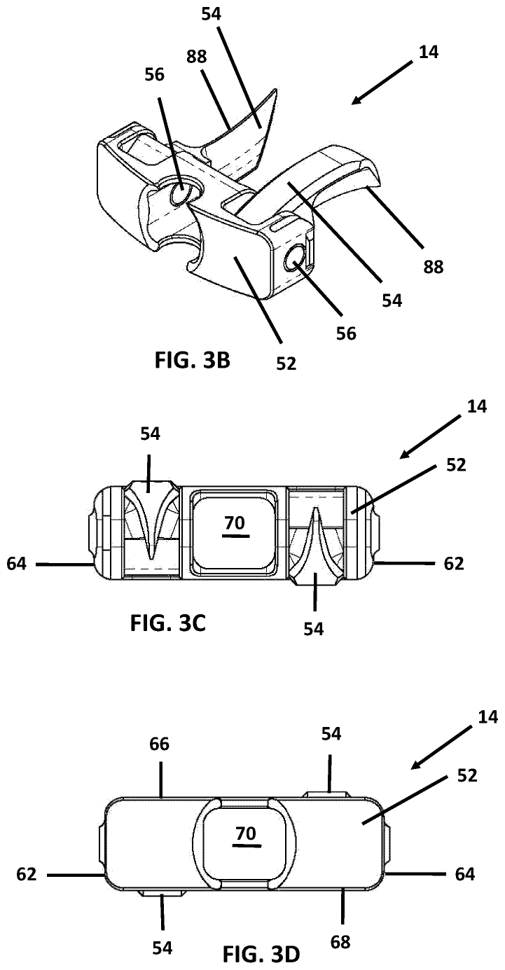

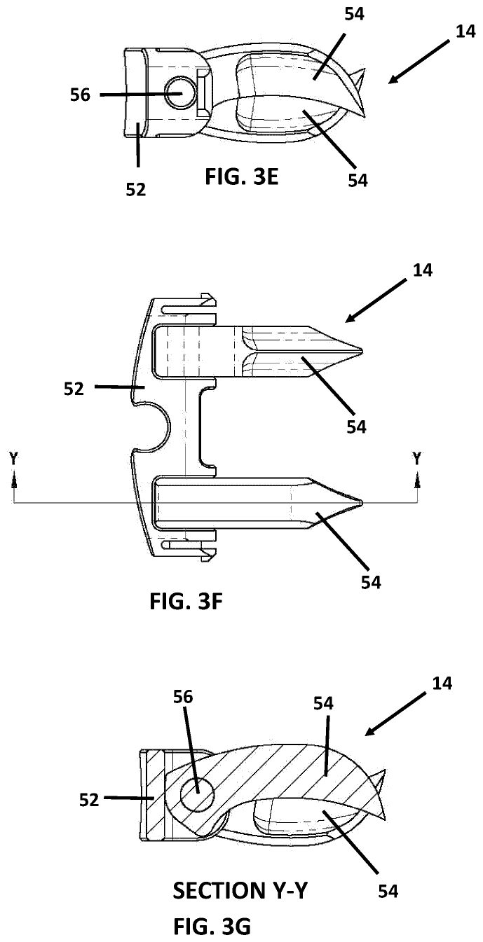

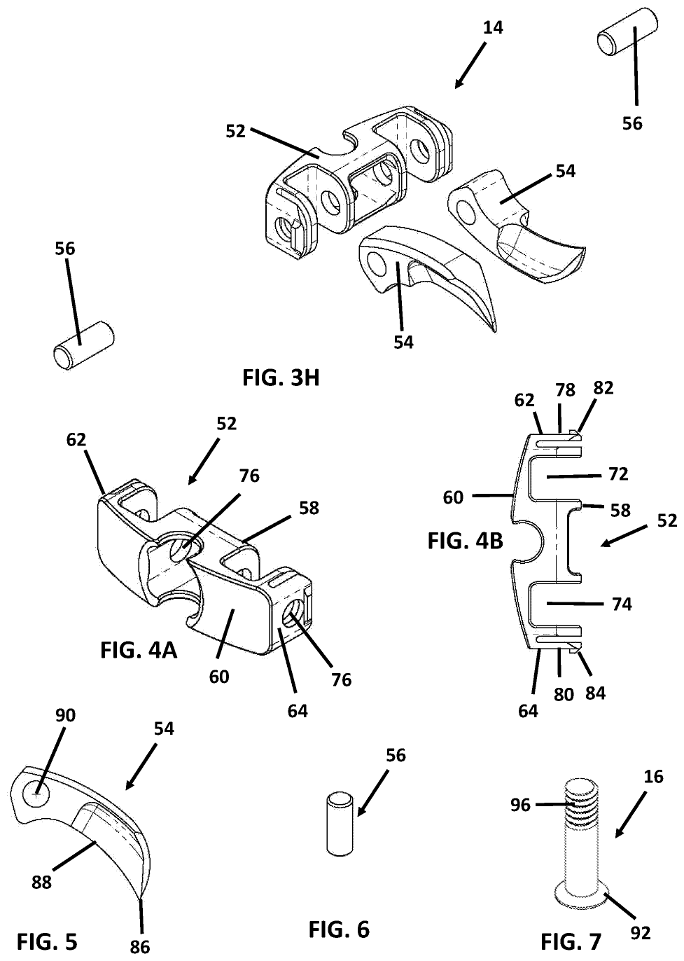

Turning now to FIGS. 3A-3H, there is shown the anchor assembly 14. The anchor assembly 14 includes a plate 52 and two bone anchors 54. The bone anchors are connected to the plate 52 with two pins 56 passed through corresponding apertures such that the bone anchors 54 are rotatable about the pins relative to the plate. The bone anchors are not removable from the plate nor are the bone anchors attached to the plate by the user. The bone anchors are attached to the plate to form a single unitary anchor assembly. The plate is further shown in FIGS. 4A-4B. The plate includes a front side 58 and a back side 60 interconnected by a left side 62, a right side 64, a top side 66 and a bottom side 68. An instrument opening 70 extends between the back side 60 and the front side 58. The instrument opening 70 has a square or rectangular shape defined by straight sides at right angles to each other. The instrument opening 70 is sized and configured to receive a correspondingly and conformingly shaped sleeve of an inserter instrument for attaching the spacer to the inserter for deployment. The front side defines a left U-shaped anchor-receiving location 72 and a right U-shaped anchor-receiving location 74. Pin apertures 76 are formed on both sides of both the left anchor-receiving location 72 and right anchor-receiving location 74. The pin apertures 76 are sized and configured to receive pins 56 passed through anchors to attach the left and right anchors 54 to the plate 52. The left side includes a left locking tab 78 having a proximal end cantilevered near the rear wall such that the left locking tab 78 flexes more at the distal end. The left locking tab 78 includes an outwardly extending left hook 82. The right side includes a right locking tab 80 having a proximal end cantilevered near the rear wall such that the right locking tab 80 flexes more at the distal end. The right locking tab 80 includes an outwardly extending right hook 84. The anchors employed in the present invention are not bone screws typically used in spinal spacers. Instead, the anchors of the present invention are curved blade or claw anchors having a sharp distal pointed tip 86, a sharp edge 88 and a pin hole 90. The convex side of the blade anchor is dull, wider and configured to slidingly ramp against the ramp surface of the cage than the opposite sharp knife edge of the blade anchor which is configured to cut into vertebral bone for entry and anchoring therein. The right anchor and left anchor are connected within the U-shaped right anchor-receiving location and left anchor-receiving location, respectively, by passing two pins through the pin apertures in the plate and the pin holes of the two anchors to attach the anchor to the plate to complete assembly of the anchor assembly. The pin-to-anchor attachment is shown to utilize two pins to attach the two anchors; however, the invention is not so limited and various attachment means are possible. The pin apertures in the plate and holes in the anchors are dimensioned with respect to the diameter of the pins such that a friction fit with frictional interference therebetween is sufficiently large so that the anchors do not flop around, rotate with respect to the plate or otherwise move under gravity or when being handled by the user. This feature advantageously maintains the anchors in an undeployed horizontal orientation so as to not interfere and remain in a low profile when manipulated by the user along the insertion pathway.

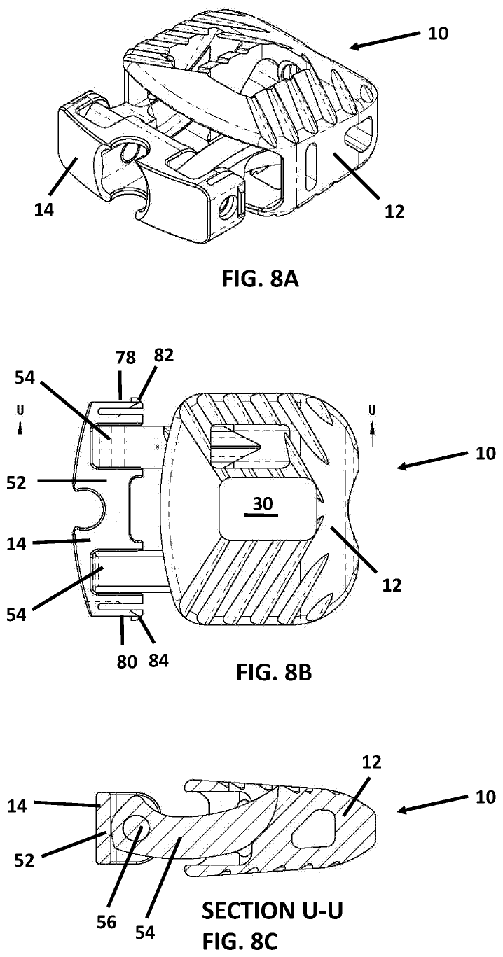

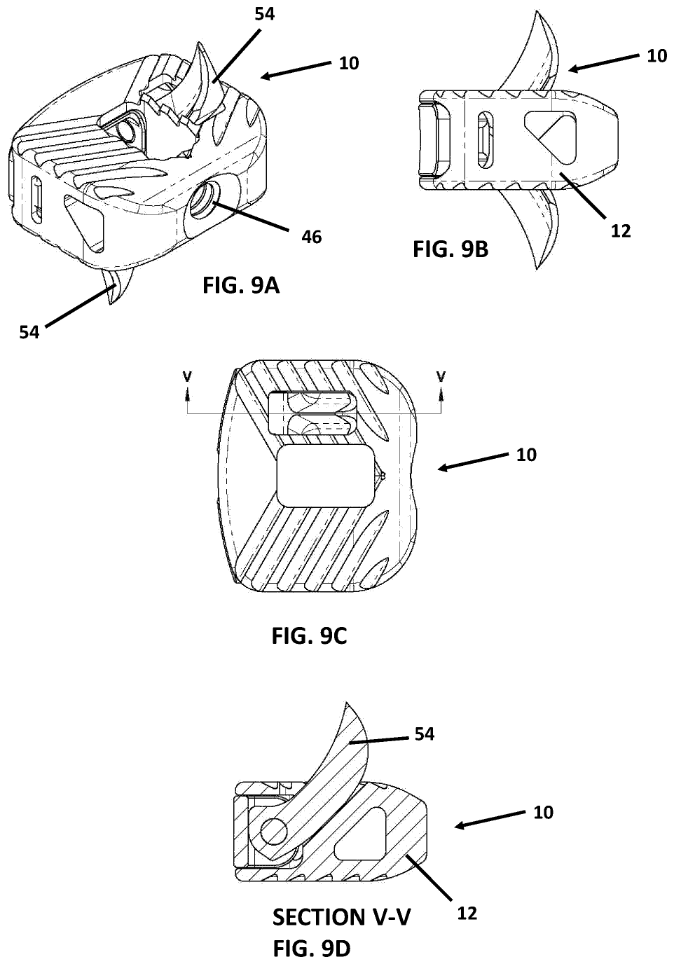

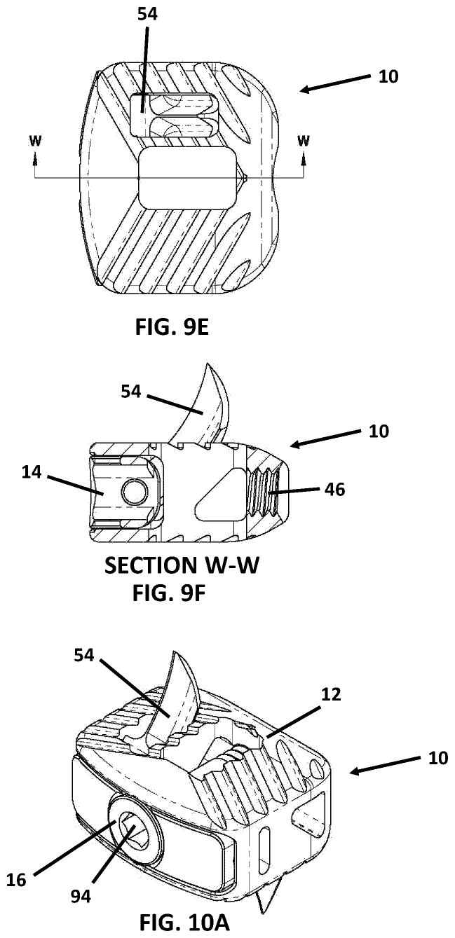

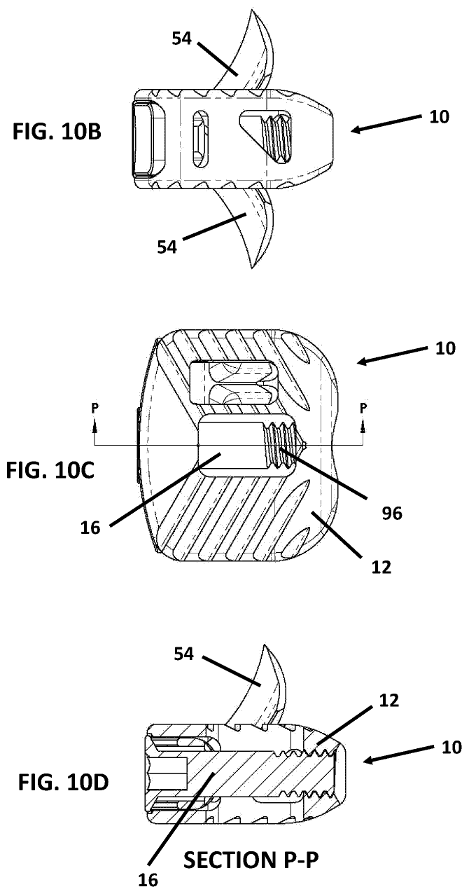

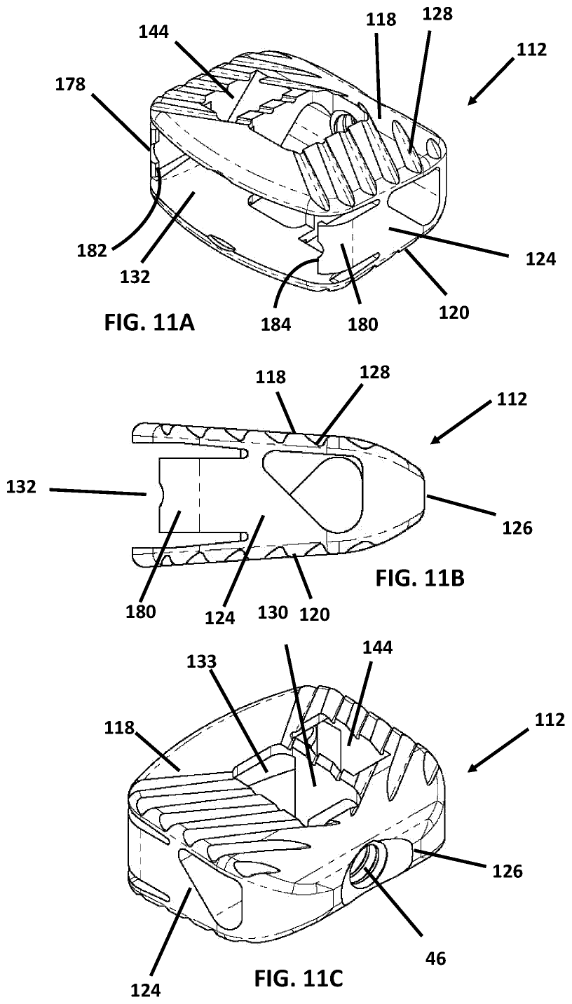

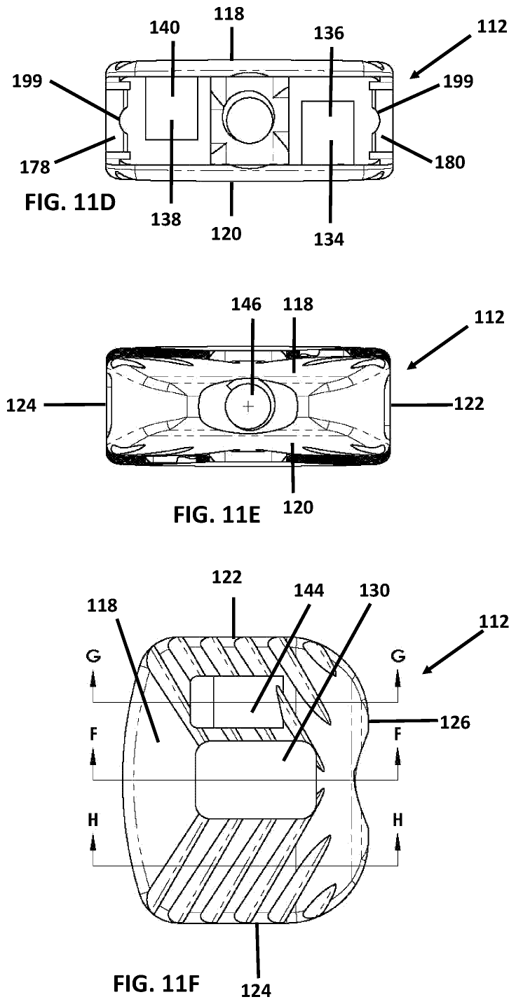

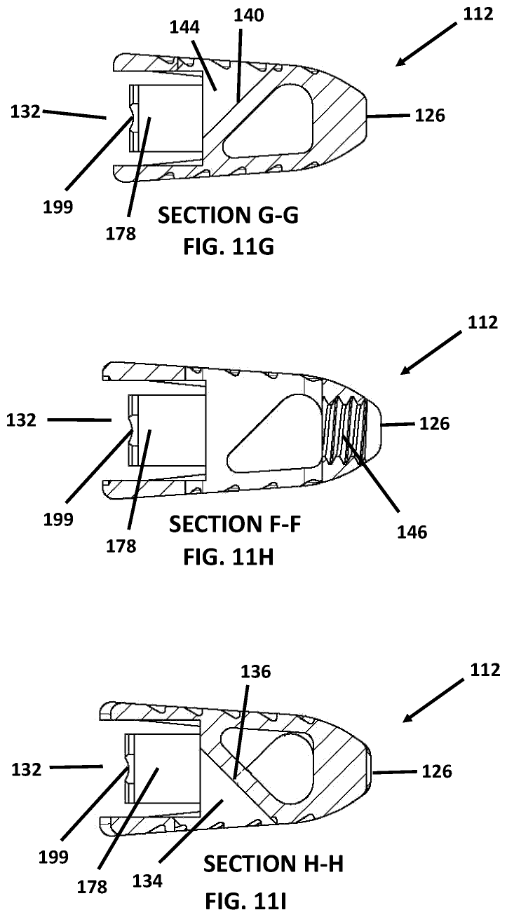

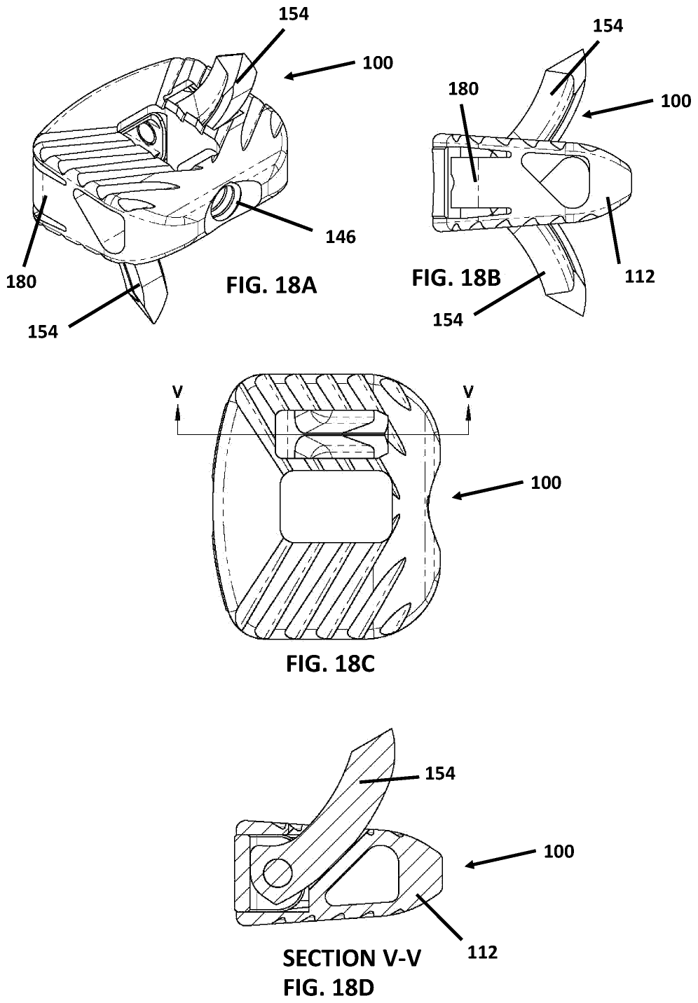

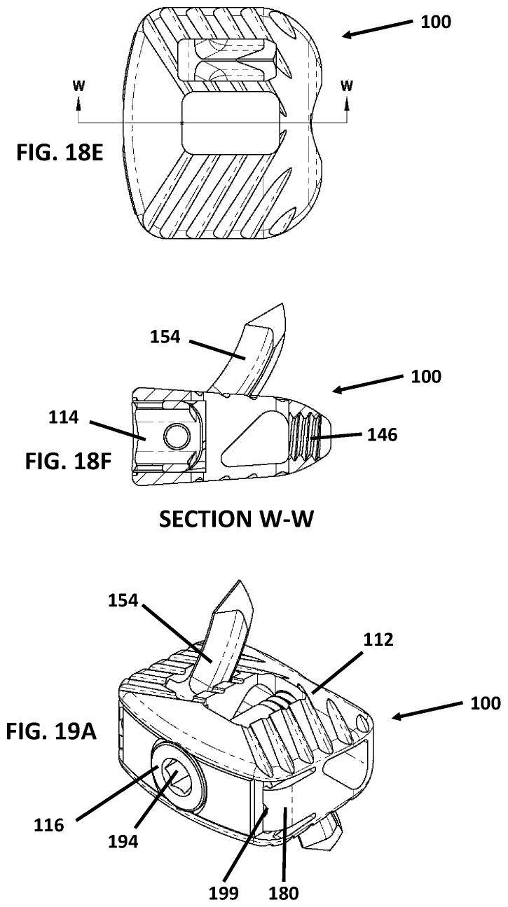

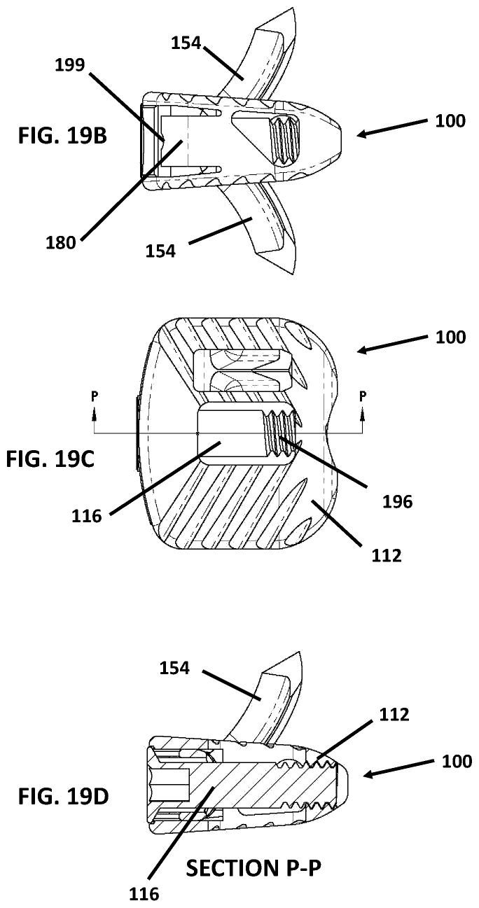

With particular reference to FIGS. 8-10, the above-described variation of the interbody spacer 10 comprises an anchor assembly removably connectable to a cage. The anchor assembly includes two non-screw, bladed anchors—one anchor configured to rotate up with respect to the plate and out of an exit opening of the cage for anchoring into an upper vertebral body and a second anchor configured to rotate down with respect to the plate and out of an exit opening of the cage for anchoring into a lower vertebral body. The cage includes a first anchor chamber having a ramped surface for directing the rotation of one anchor upwardly and a second anchor chamber having a ramped surface for directing the rotation of the second anchor downwardly. The anchor assembly is configured to be inserted anchor first in through the proximal opening of the cage. Forward translation of the anchor assembly with respect to the cage for insertion of the anchor assembly into the cage causes the distal ends of the anchors to contact their respective ramped surface and with continued forward translation cause them overcome any friction with respect to the plate and to rotate with respect to the plate about their pins to exit such that one anchor projects into an upper vertebral body and the second anchor projects into a lower vertebral body when located within a disc space. Further distal translation of the anchor assembly causes the left and right locking tabs on either side of the plate to flex inwardly when the plate is inserted into the cage. The locking tabs have an angled tip that assists the insertion such that the angled tip ramps against the inner surface of the left and right sidewalls of the cage. The left locking tab will deflect and then snap back outwardly when the left locking tab is no longer retained in a deflected state in the location of the left locking slot into which the left hook will snap into. Similarly, the right locking tab will deflect and then snap back outwardly when the right hook snaps into the right locking slot. The hooks have an angled distal end that aids insertion and deflection of the locking tabs. The hooks have a vertical abutment at the proximal end that prevents the anchor assembly from backing out of the cage to lock the anchor assembly to the cage. The flexible locking tabs create a snap fit engagement into the slots of the cage to lock the anchor assembly to the cage. The anchor assembly may be removed by employing an instrument inserted into the left and right locking slots to deflect the locking tabs inwardly to back the anchor assembly out of the cage. A secondary lock 16 depicted in FIG. 7 may be inserted into the instrument opening on the plate. The secondary lock is configured as a screw having a flat head 92 with a socket 94 and a distally threaded shaft 96. When the secondary lock is inserted in through the instrument opening the distal threaded shaft will threadingly engage with the distal threaded opening of the cage. The secondary lock is screwed to the cage capturing the locking assembly between the head and the cage to provide secondary backout protection for the anchor assembly in addition to the primary backout protection provided by the snap lock action of the locking tabs. This variation of the spacer describes backout protection designed with deflectable locking tabs on the plate that spring lock into slots on the cage. Next, a variation of the spacer in which the deflectable locking tabs are on the cage that spring lock over a protruding ledge located on the plate will be described in reference to FIGS. 11-19.

Turning now to FIGS. 11-19, a variation of an interbody spacer 100 will be described in which like reference numbers will be used to describe like parts or where the same reference numbers will be used preceded by “1” to place all the reference numbers for the second variation in the 100s with their corresponding description appearing above. Furthermore, only the differences over the previous variation will be described hereinbelow as the similarities of structure and function are evident to one skilled in the art from the previous description.

With particular reference to FIGS. 11A-11I, a cage 112 is shown wherein the left sidewall 122 is provided with a left locking tab 178 cantilevered at the distal end to the left sidewall such that the proximal end of the left locking tab is capable of flexing a greater distance relative to the distal end. The right sidewall 124 is provided with a right locking tab 180 cantilevered at the distal end to the right sidewall such that the proximal end of the right locking tab is capable of flexing a greater distance relative to the distal end. The left locking tab includes a left hook 182 located at the proximal end of the locking tab seen in FIG. 11A. The left hook extends inwardly as can be seen in FIG. 11D. The right locking tab includes a right hook 184 located at the proximal end of the locking tab seen in FIG. 11A. The right hook extends inwardly as can be seen in FIG. 11D. Still referencing FIG. 11D, the left and right hooks include a curved scalloped indentation 199 sized and configured to receive an instrument for disengaging the locking tabs in order to remove the anchor assembly 114 from the cage 112.

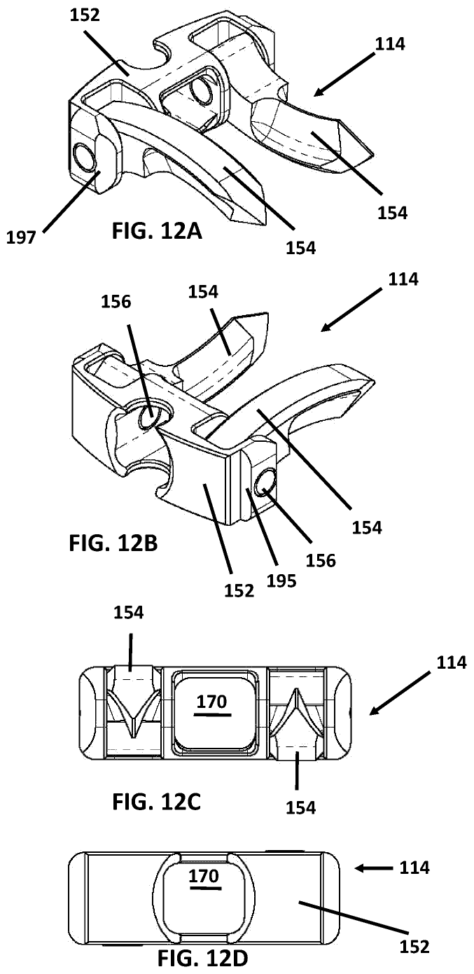

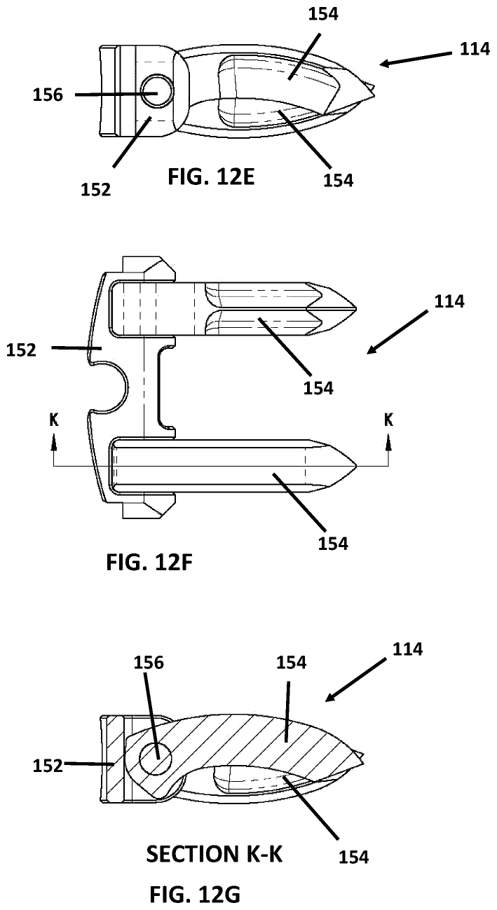

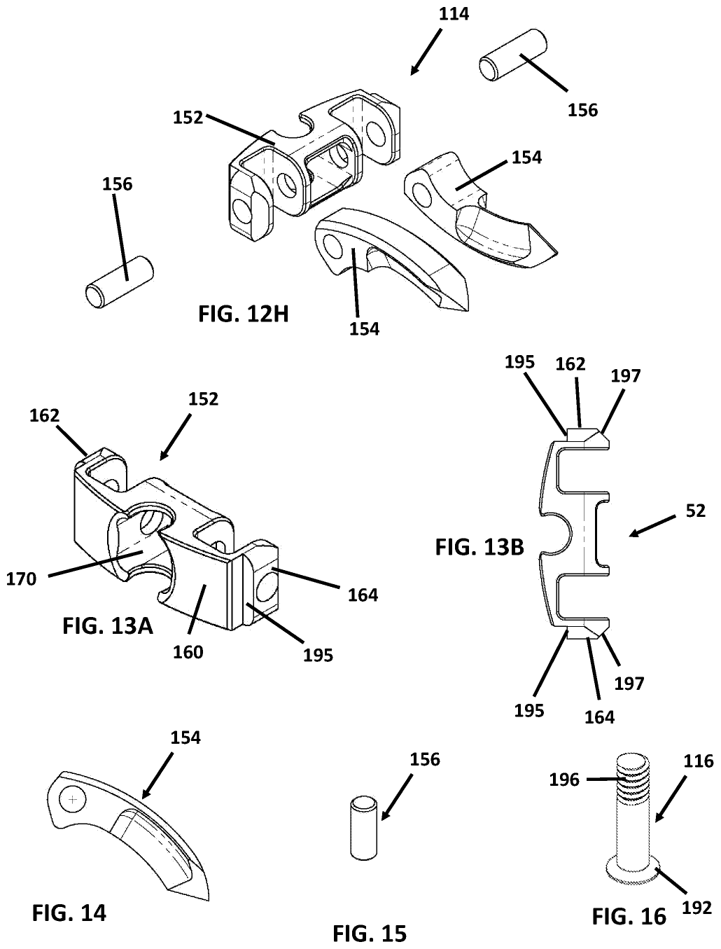

With reference now to FIGS. 12-13, the differences of the plate 152 will now be described. The left side 162 and right side 164 of the plate 152 each include an angled distal end 197 and a proximal ledge 195 sized and configured to engage the left locking tab and right locking tab, respectively, so as to deflect the locking tabs outwardly to ramp along the outer surface of the left side and right side and then to spring back inwardly to lock and abut against the ledge 195. This snap back action of the cage locking tabs provides a primary anti-backout protection preventing the anchor assembly from backing out of the cage.

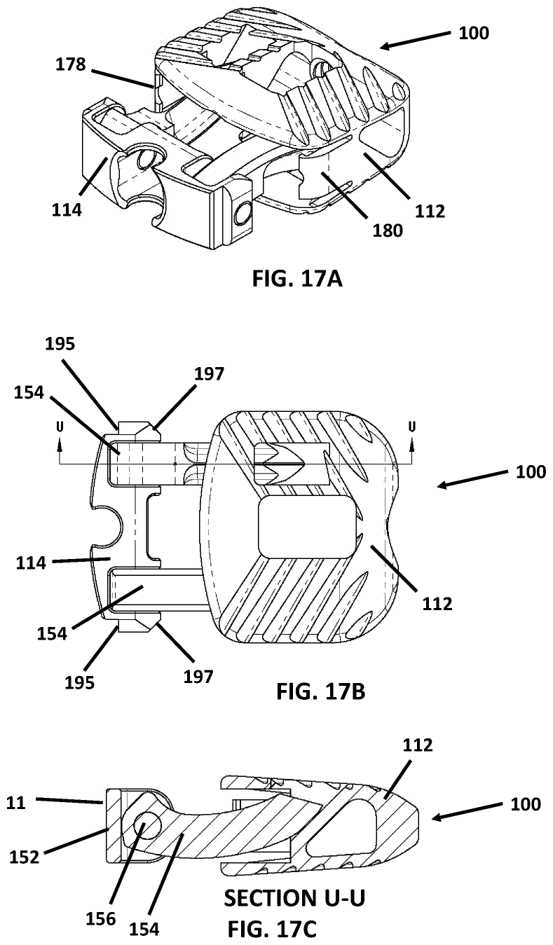

With particular reference to FIGS. 17-19, distal translation of the anchor assembly, causes the left and right locking tabs 178, 178 on either side of the plate to flex inwardly when the plate is inserted into the cage. Each of the locking tabs has an angled proximal end that assists the insertion such that the angled proximal end ramps against the angled distal end 197 on the plate 152. The left locking tab on the cage will deflect outwardly and then snap back inwardly when the left locking tab has ramped over the ledge 195 on the left side of the plate 152. Similarly, the right locking tab on the cage will deflect outwardly and then snap back inwardly when the right locking tab has ramped over the ledge 195 on the right side of the plate 152. The flexible locking tabs on the cage create a snap fit engagement onto the sides of the plate to lock the anchor assembly to the cage. A secondary lock 16 depicted in FIG. 7 may be inserted into the instrument opening on the plate. The secondary lock 116 is configured as a screw having a flat head 192 with a socket 194 and a distally threaded shaft 196. When the secondary lock is inserted in through the instrument opening the distal threaded shaft will threadingly engage with the distal threaded opening of the cage. The secondary lock is screwed to the cage capturing the locking assembly between the head and the cage to provide secondary backout protection for the anchor assembly in addition to the primary backout protection provided by the snap lock action of the locking tabs. This variation of the spacer describes backout protection designed with deflectable locking tabs on the cage that spring lock onto the opposite sides of the plate.

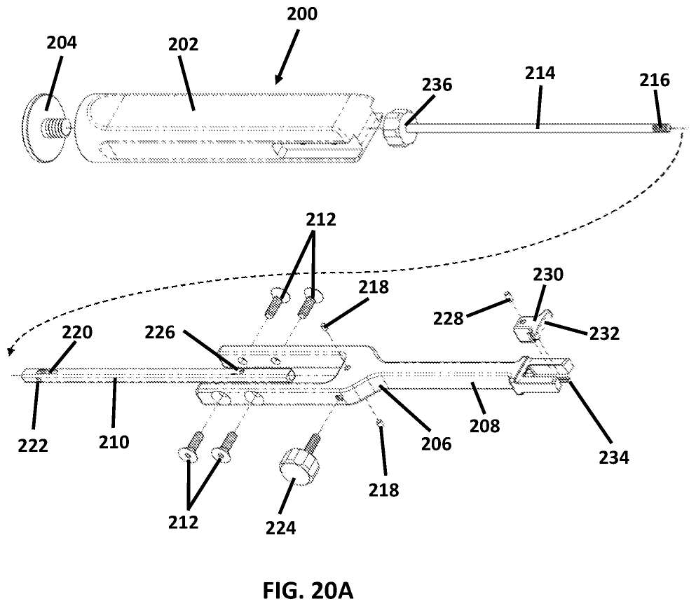

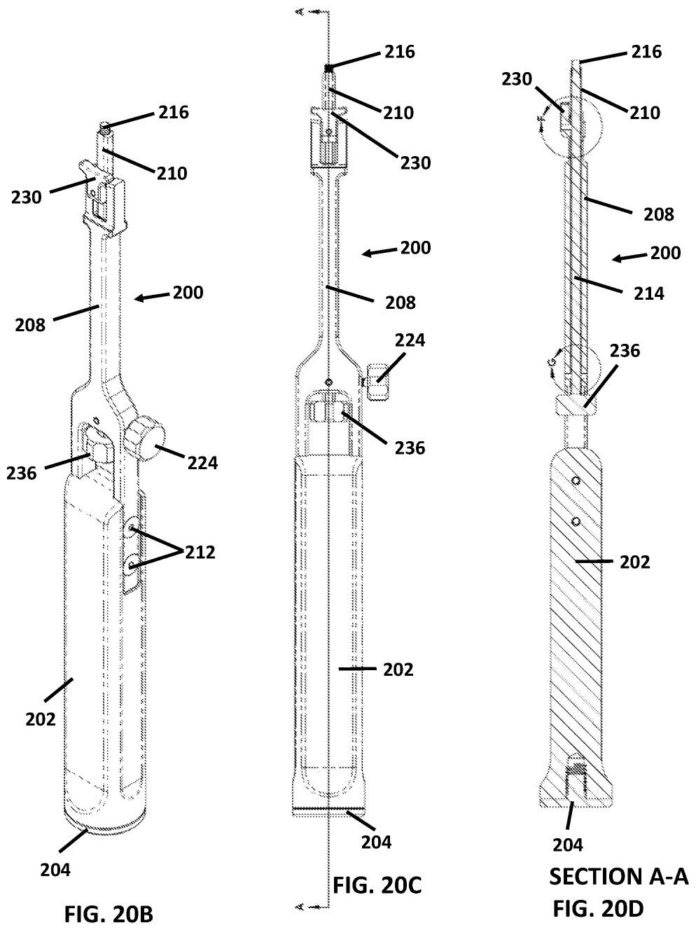

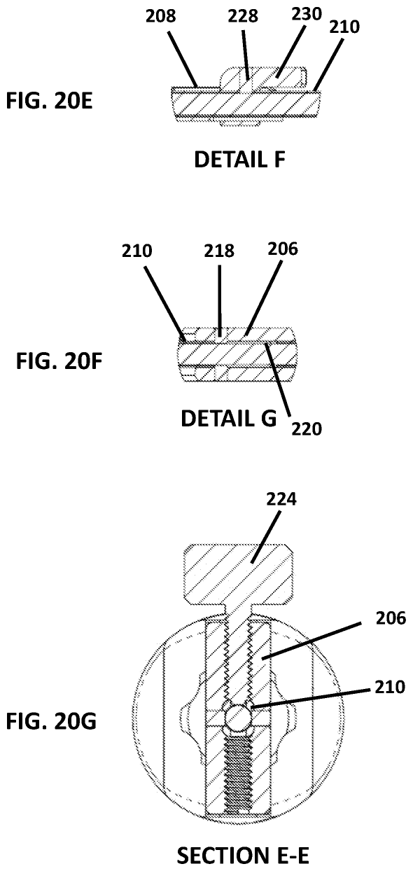

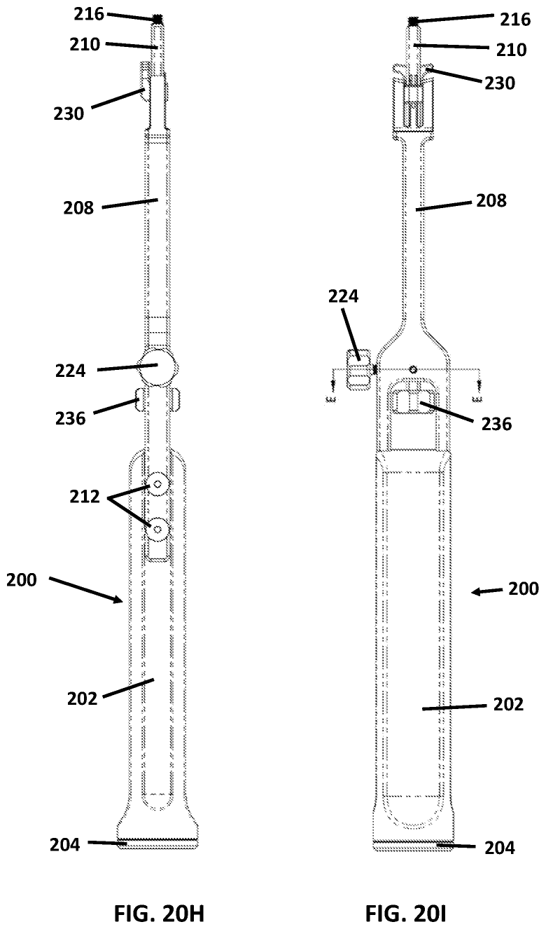

Turning now to FIG. 20, the inserter instrument 200 will now be described. The inserter includes a handle 202 having a stainless-steel endcap 204 at the proximal end. A main body 206 is connected to the distal end. The main body has a forked proximal end and forked distal end. The central shaft 208 of the main body is rectangular in shape and has a central inner channel sized that is configured to receive a hollow sleeve 210 that is also rectangular in shape. The main body is connected to the handle by four flat head screws 212 to capture the sleeve and to capture an internal shaft 214 between the main body and handle such that the internal shaft can rotate relative to the handle, main body and sleeve. The internal shaft is provided with a proximal knob 236 for the user to rotate the internal shaft. The internal shaft has a threaded distal end 216 that is sized and configured to threadingly engage with the threaded opening on the cage. The longitudinal translation of the sleeve is limited by a stop pin 218 inserted into the main body and into a slot 220 on the sleeve. The length of the slot on the sleeve serves as a proximal and distal abutment for the stop pin inserted therein to limit the longitudinal translation of the sleeve. Also, at the proximal end of the sleeve a hole 222 is provided that is sized and configured to receive the distal end of a lock 224. The lock is threaded into an aperture on the main body. There is a boss feature on the tip of the lock which goes into a hole on the sleeve which serves to lock the sleeve to the main body preventing the main body from moving relative to the sleeve. The distal end of the sleeve is provided with a depth stop pin hole 226 for receiving a depth stop pin 228. The depth stop pin is used to connect a U-shaped depth stop 230 to the sleeve. The depth stop has a distal surface 232 located on one side of the forked distal end of the main body so that the distal surface may contact against one of the upper or lower vertebral bodies to limit the depth of insertion of the spacer within the disc space. Furthermore, tangs 234 are provided to retain the bone anchor assembly to the inserter. FIGS. 20B-20I illustrate the starting position of the inserter. The starting position also includes the lock 224 being engaged into the sleeve. This means that the boss on the tip of the lock is in the hole of the sleeve. The user will know if this is the case if the main body does not slide relative to the sleeve.

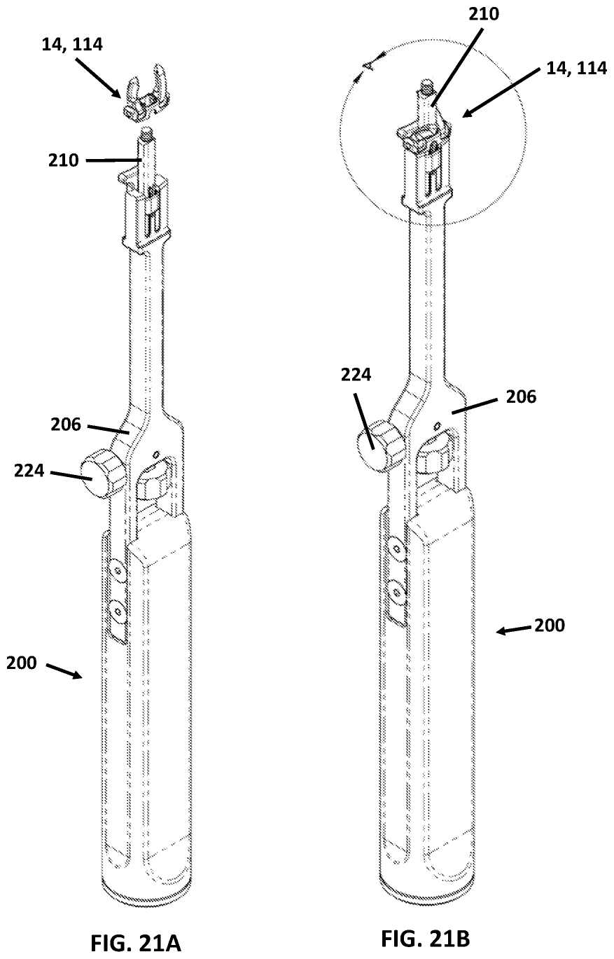

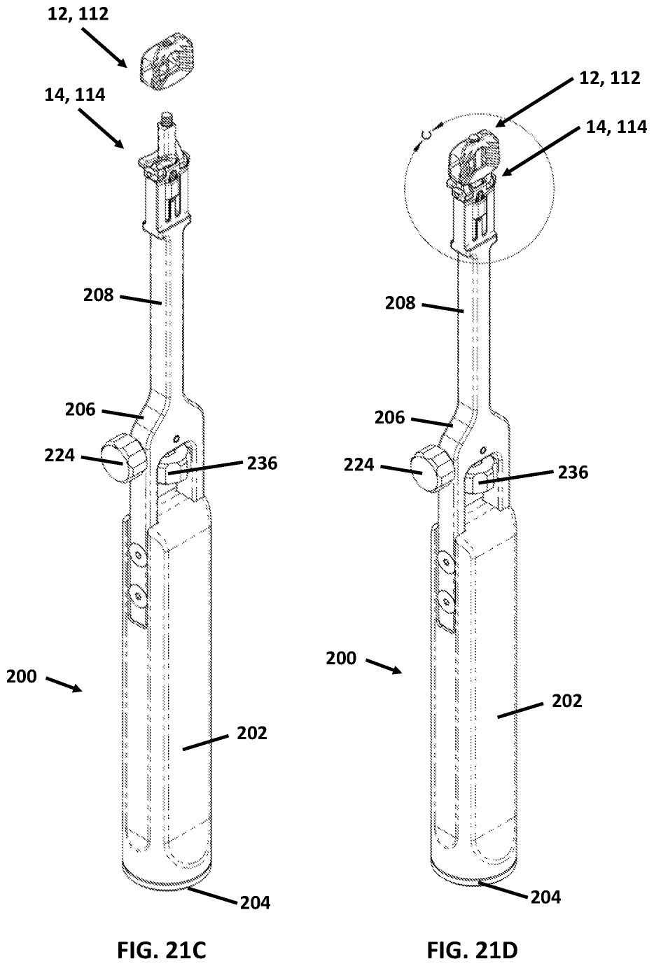

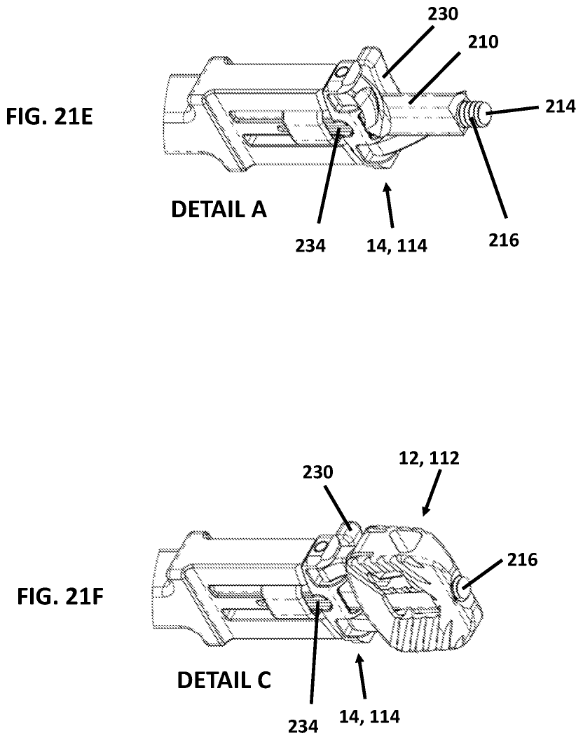

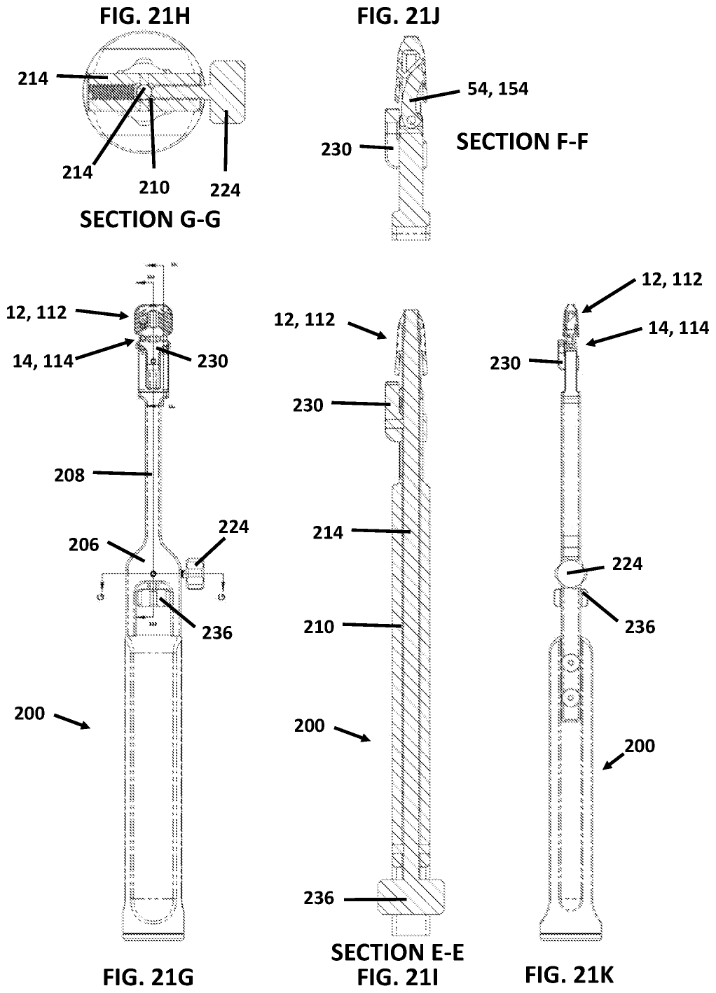

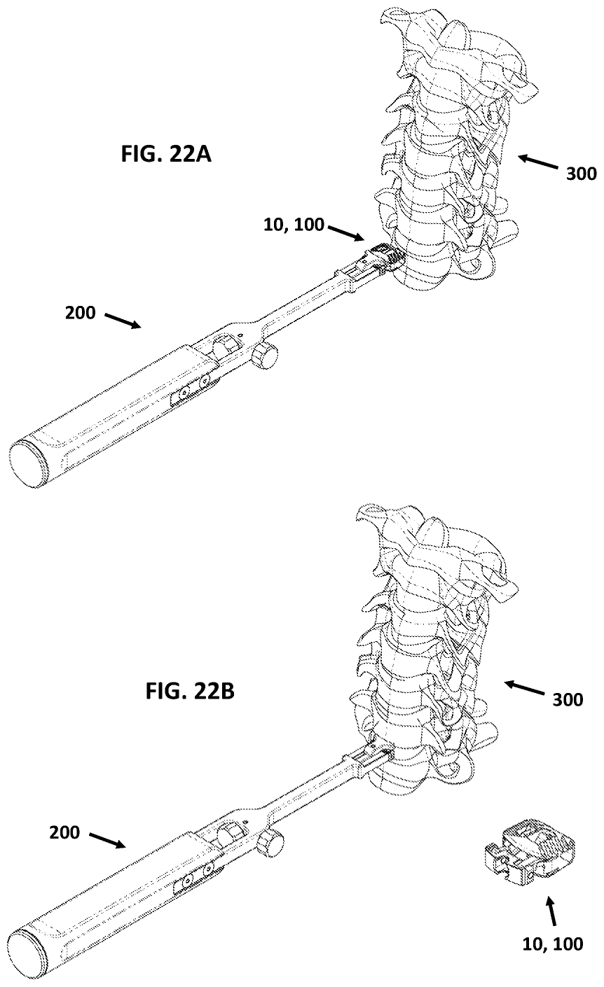

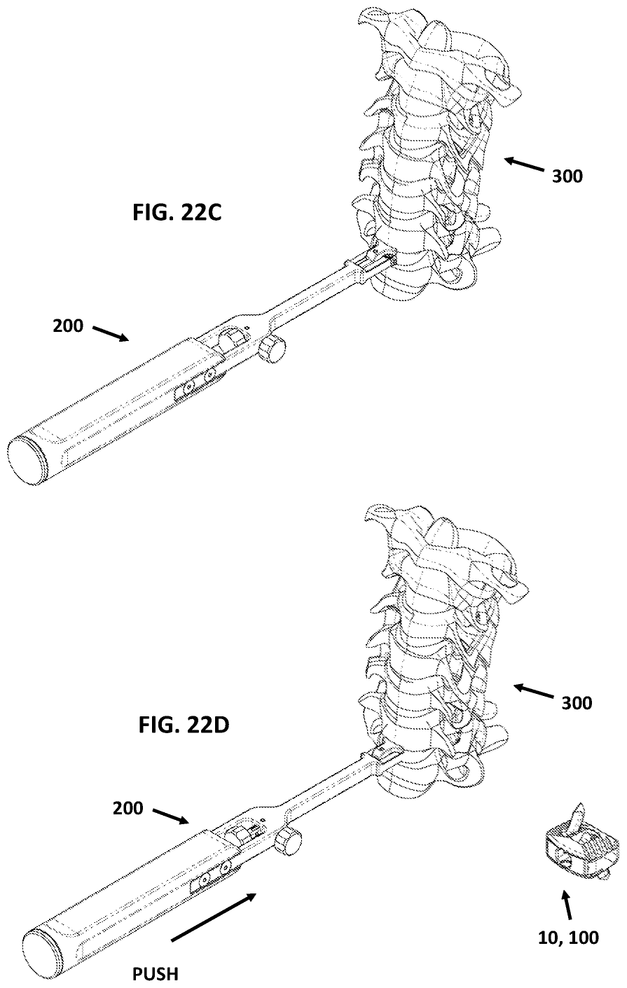

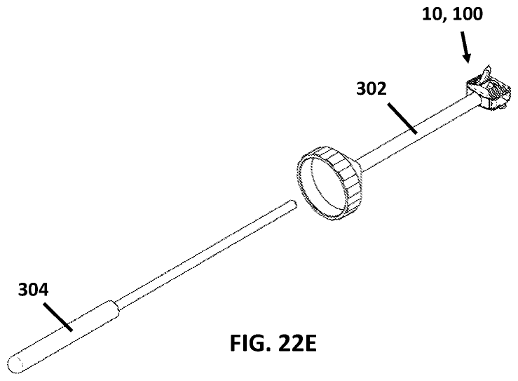

Turning now to FIGS. 21A-21B and 21E, from the starting position of FIGS. 20B-20I of the inserter, the loading of the anchor assembly 14, 114 and cage 12, 112 onto the inserter 200 will now be described. Firstly, if the lock 224 is not engaged, the lock 224 on the right side of the inserter is rotated to pin the internal shaft 214 to fix its position and the position of the sleeve 210 with respect to the main body 206 as shown in FIG. 21H. The anchor assembly 114 slides over the sleeve 210 as seen in FIGS. 21B and 21E. In particular, the rectangular sleeve 210 is inserted into the rectangular instrument opening of the plate 70, 170. Tangs 234 are provided at the distal end of the inserter 200 and located between the distal fork of the main body 206 as can be seen in FIG. 21E. When the anchor assembly 14, 114 is passed over the sleeve 210 of the inserter, the rounded tangs 234 are easily deflected inwardly toward each other at the neck of the circular opening at the bottom end of the plate 52, 152. The tangs then snap into the circular opening and because of their conforming shape, the tangs connect and retain the anchor assembly to the inserter. Next, the cage 12, 112 slides over the sleeve 210 as shown in FIGS. 21C, 2I D, 21F. In particular, the rectangular sleeve 210 is inserted into the rear opening 32, 132 of the cage 12, 112. The rectangular window 33, 133 on the cage advantageously and easily places the cage into the proper orientation with respect to the inserter instrument. The internal shaft 214 is rotated by rotating the knob 236 at the proximal end of the internal shaft. Rotation of the internal shaft threads the distal end 216 of the internal shaft into the threaded opening of the cage 46, 146. This secures the cage to the inserter. Next, the lock 224 on the right side of the inserter is rotated to pin the internal shaft 214 to fix its position and the position of the sleeve 210 with respect to the main body 206 as shown in FIG. 2I H. Next, the spinal column 300 and target disc space is approached by the inserter 200 loaded with the attached anchor assembly 14, 114 and cage 12, 112 as shown in FIG. 22A. The disc space has already been prepped to receive the implant and the interbody spacer is inserted into the target disc space as shown in FIG. 22B until the depth stop surface 232 on the depth stop 230 contacts the upper vertebral body as shown. Of course, the inserter may be designed with the depth stop on the bottom and the tangs oppositely located on the top in which case, the depth stop surface would contact the lower vertebral body. In FIGS. 22B-22D, the interbody spacer 10, 100 cannot be seen as it is located within the intervertebral space. For illustrative purposes, FIGS. 22B-22D include a depiction of interbody spacer alongside the spinal column 300. The interbody spacer is shown in its current state of deployment corresponding to the step of insertion depicted in the figure therein. Next, the lock 224 is released by rotating the lock 224 on the right side in the opposite direction as shown in FIG. 22C. This frees the sleeve and internal shaft to move relative to the main body. Next, the user will use a mallet to hammer the steel endcap 204 at the proximal end of the handle 202 to drive the anchor assembly forward as shown in FIG. 22D. As the user hammers the proximal end of the inserter, the distal end of the forked main body will contact the plate and move it forward relative to the cage advancing the anchor assembly into the cage into a deployed configuration in which the anchors ramp against the ramp surfaces and exit the cage and enter the bony material of the vertebrae anchoring the spacer in the disc space with the primary anti-backout locking tabs engaged in a locked position as described above. The inserter be used with either of the variations of the interbody spacer 10, 100 described above. The inserter is released from the cage by unscrewing the inner shaft by rotating knob 236. The inserter is pulled in the proximal direction and tangs deflect inwardly and release from the plate. With the inserter removed from the spacer, a bone graft funnel 302 can be attached to the spacer 10, 100 as shown in FIG. 22E. Bone graft material is inserted into the funnel and tamped down with a bone graft tamp 304 also shown in FIG. 22E. Bone graft is easily delivered through the instrument opening in the plate, through the rectangular window and into the central opening of the cage. An optional secondary lock 16, 116 may be inserted to keep the anchor assembly attached to the cage as described above. The secondary lock 16, 116 includes a socket in the head for attachment to a driver.

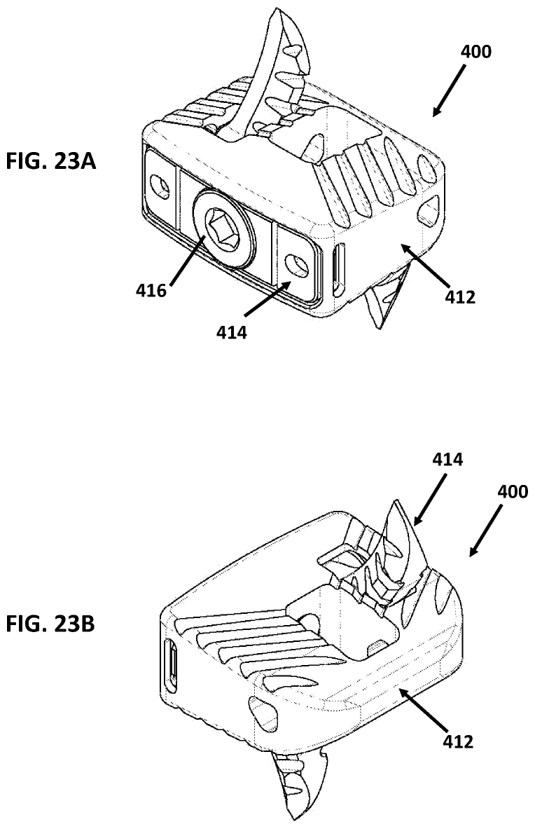

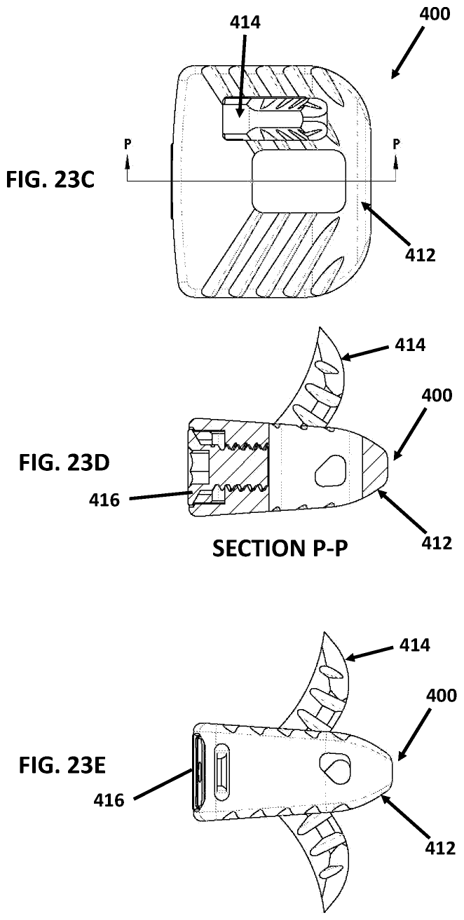

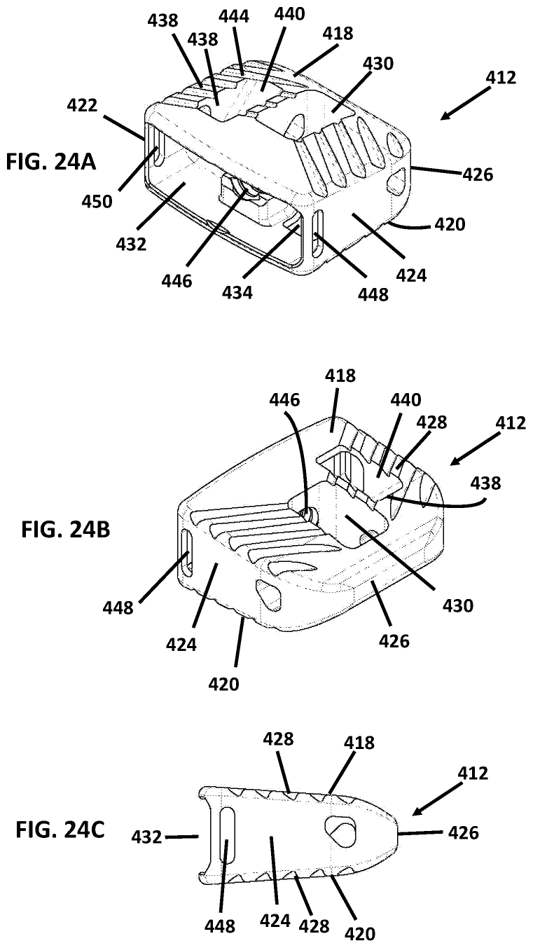

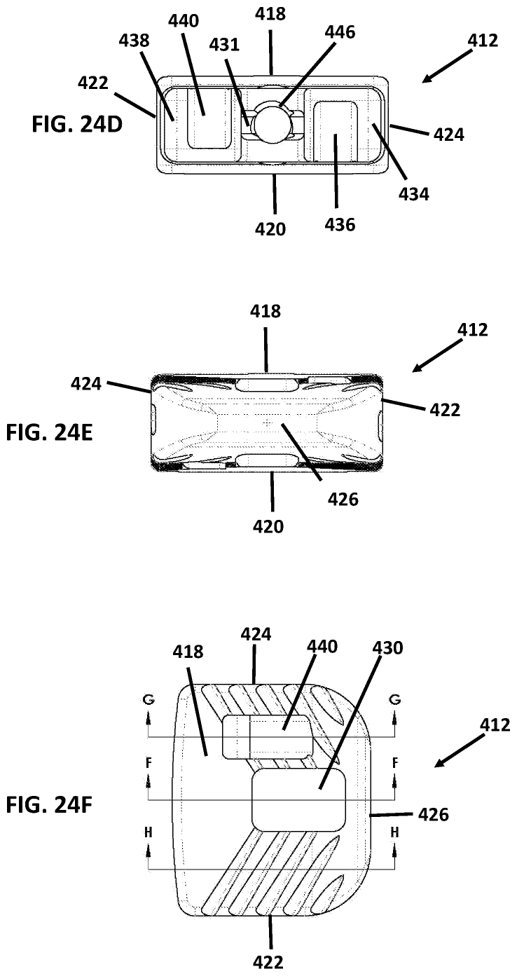

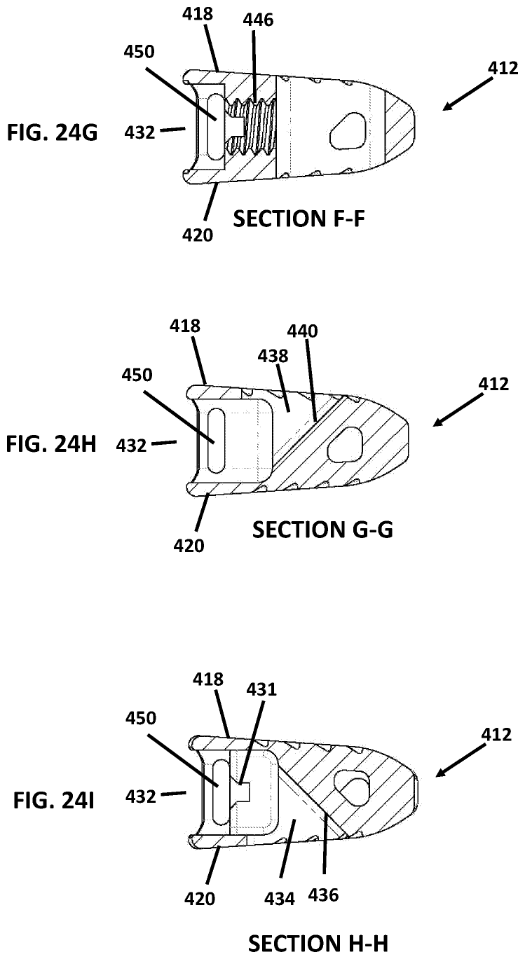

Turning to FIGS. 23A-23E, there is shown another variation of an interbody spacer 400. The present invention describes an interbody spacer 400 comprising a cage 412 removably connectable to an anchor assembly 414 with an optional secondary lock 416 as shown in FIG. 23A. The cage 412 by itself is shown in FIGS. 24A-24I. The cage 412 includes a top endplate 418 and a bottom endplate 420 interconnected by a left sidewall 422, a right sidewall 424, and a leading end wall 426. The spacer 400 does not have a trailing end wall. Instead, the trailing end of the spacer 400 is provided with a rectangular-shaped rear opening 432 that extends between the top endplate and bottom endplate and between the left sidewall and right sidewall. The top endplate and bottom endplate each have ridges 428 for increasing traction against two adjacent vertebral bodies between which the spacer 400 is inserted. The cage 412 includes a central opening 430 extending vertically through the cage 412 between the top endplate and the bottom endplate. The central opening decreases the weight of the spacer and provides a location for bone graft placement for bone ingrowth to occur to further stabilize the spacer 400 with respect to the spine. The rear opening 432 is interconnected with the central opening 430 via a threaded bore 446 that lies along the central horizontal longitudinal axis of the spacer 400. The threaded bore 446 is provided a distance from the rear end. Leading into the threaded bore 446 is a horizontally oriented slot 431 extending radially across the threaded bore 446. The slot 431 is sized and configured to engage with an insertion instrument and for orientating it with respect to the cage 412. The central opening 430 is interconnected with the threaded bore 446. Bone graft may be inserted through the rear opening 432, and/or central opening 430 after implantation of the spacer 400 in the vertebral space or prior to implantation. The cage 412 further includes a right anchor chamber 434 having a right ramped surface 436 and a left anchor chamber 438 having a left ramp surface 440. The cage 412 includes a right chamber exit opening 442 formed in the bottom endplate 420 and a left chamber exit opening 444 formed in the top endplate 418. The right exit opening 442 is rectangular in shape and defines the floor of the right chamber being in communication therewith. The left exit opening 444 is rectangular in shape and is defined in the ceiling of the left anchor chamber 438. The right sidewall defines a vertically extending locking slot 448 and the left sidewall defines a vertically extending locking slot 450. The right ramp surface 436 is angled downwardly to meet the front end of the right exit opening 442. The left ramp surface 440 is angled upwardly to meet the front end of the left exit opening 444.

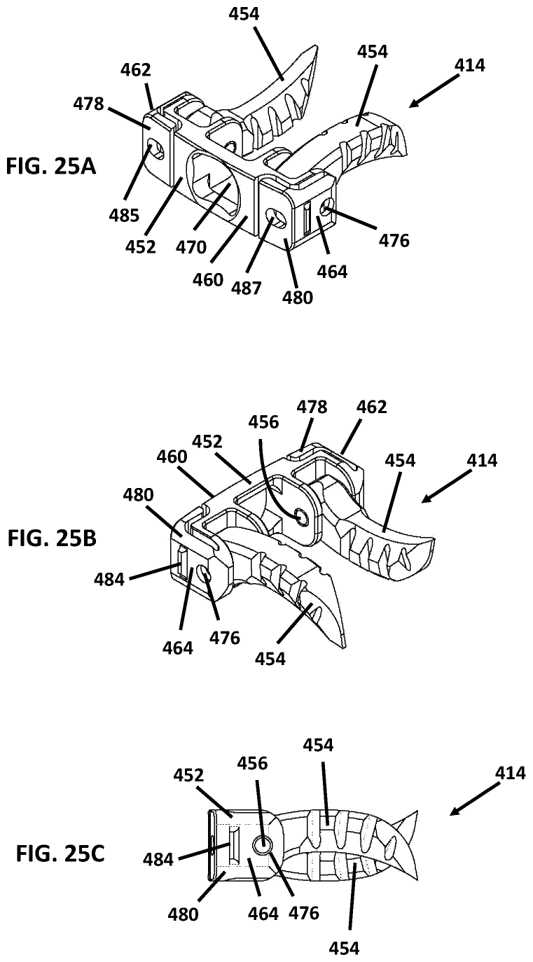

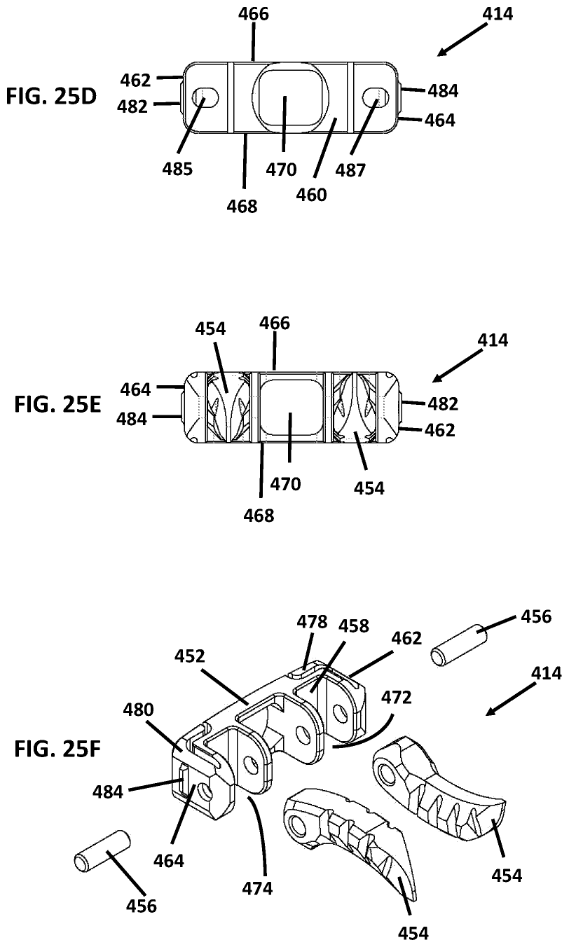

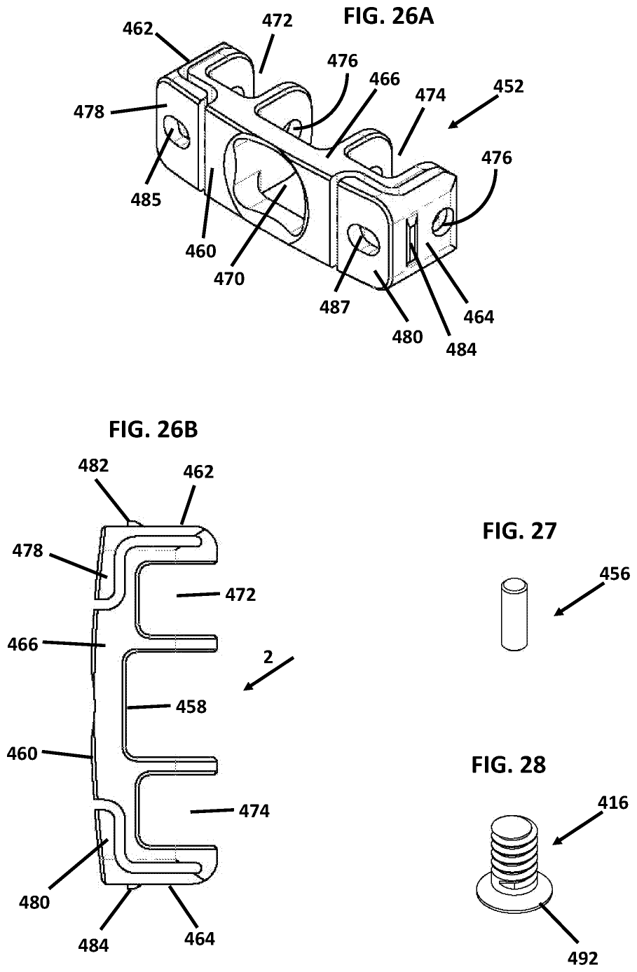

Turning now to FIGS. 25A-25F, there is shown the anchor assembly 414. The anchor assembly 414 includes a plate 452 and two bone anchors 454. The bone anchors are connected to the plate 452 with two pins 456 passed through corresponding apertures such that the bone anchors 454 are rotatable about the pins 456 relative to the plate 452. The plate 452 is further shown in FIGS. 26A-26B. The plate 452 includes a front side 458 and a back side 460 interconnected by a left side 462, a right side 464, a top side 466 and a bottom side 468. An instrument opening 470 extends between the back side 460 and the front side 458 of the plate. The instrument opening 470 has a square or rectangular shape defined by straight sides at right angles to each other but may be of any shape that conforms with the shape of inserter instrument to help orient the instrument with respect to the anchor assembly 414. The instrument opening 470 is sized and configured to receive a correspondingly and conformingly shaped sleeve of an inserter instrument for attaching the spacer 400 to the inserter for deployment. The front side defines a left U-shaped anchor-receiving location 472 and a right U-shaped anchor-receiving location 474. Pin apertures 476 are formed on both sides of both the left anchor-receiving location 472 and right anchor-receiving location 474. The pin apertures 476 are sized and configured to receive pins 456 passed through anchors to attach the left and right anchors 454 to the plate 452. The left side includes a left locking tab 478 having a distal end cantilevered near the front wall 458 such that the left locking tab 478 flexes more at the proximal end. The left locking tab 478 includes an outwardly extending left hook 482. The right side includes a right locking tab 480 having a distal end cantilevered near the front wall 458 such that the right locking tab 480 flexes more at the proximal end. The right locking tab 480 includes an outwardly extending right hook 484. The left locking tab 478 is L-shaped when viewed from the top having the top of the “L” connected to the plate 452 and the bottom of the “L” free to flex. The L-shaped locking tab is formed in the entire plate 452. The rear-facing side of the left locking tab 478 or the bottom leg of the “L” includes an aperture 485. The aperture 485 is circular or elliptical in shape. The elliptical/oblong shaped aperture 485 allows the left locking tab 478 to move with respect to inserted locking tabs of the inserter instrument. As the left locking tab 478 flexes with respect to the plate, the left locking tab 478 is free to move with respect to the inserter instrument locking tabs inserted therein. The left locking tab 478 is formed by an L-shaped cut vertically through the plate from the top side 466 to the bottom side 468 of the plate 452. The right locking tab 480 is L-shaped when viewed from the top having the top of the “L” connected to the plate 452 and the bottom of the “L” free to flex. The L-shaped locking tab is formed in the entire plate 452. The rear-facing side of the right locking tab 480 or the bottom leg of the “L” includes an aperture 487. The aperture 487 is circular or elliptical in shape. The elliptical/oblong shaped aperture 487 allows the right locking tab 480 to move with respect to inserted locking tabs of the inserter instrument. As the right locking tab 480 flexes with respect to the plate, the right locking tab 480 is free to move with respect to the inserter instrument locking tabs inserted therein. The right locking tab 480 is formed by an L-shaped cut vertically through the plate from the top side 466 to the bottom side 468 of the plate 452. Both apertures 485, 487 face the back side 460 of the plate and extend longitudinally distally through their respective locking tabs 478, 480.

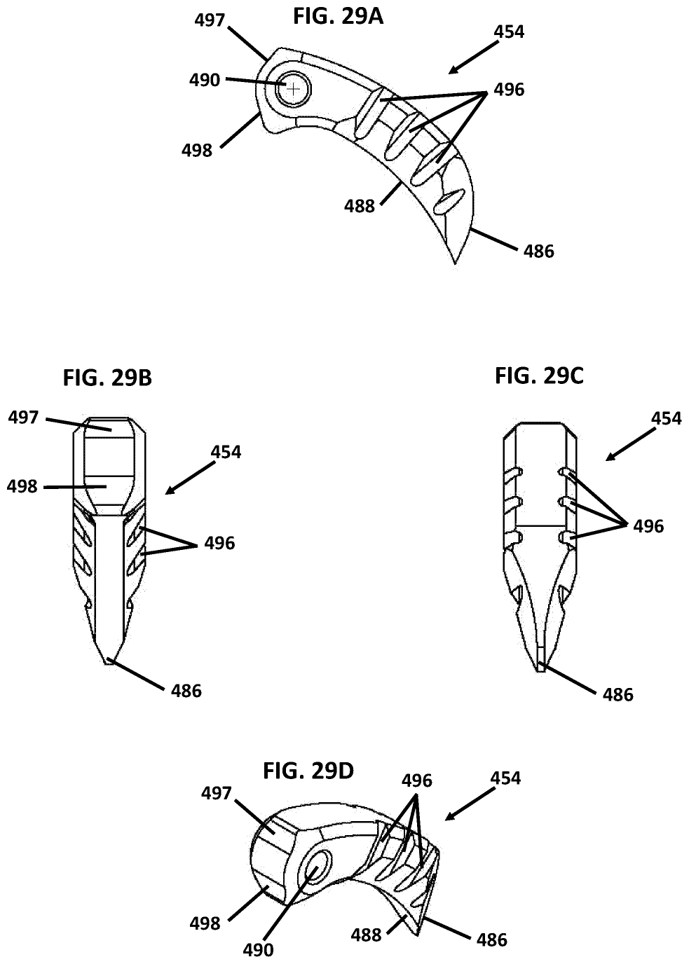

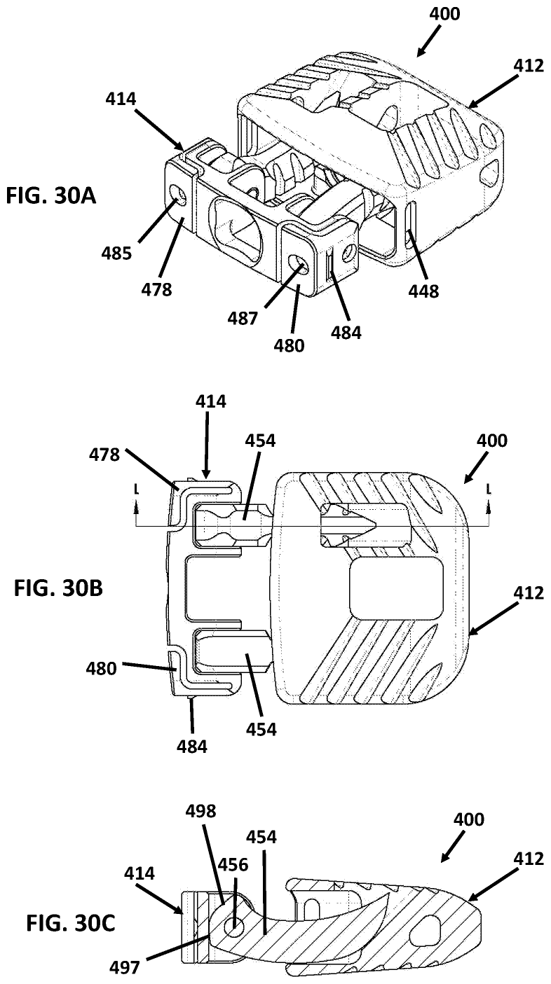

Turning to FIGS. 29A-29D, the anchors 454 employed in the present invention are not bone screws typically used in spinal spacers. Instead, the anchors of the present invention are curved blade or claw anchors having a sharp distal pointed tip 486, a sharp edge 488 and a pin hole 490. The convex side of the blade anchor is dull, wider and configured to slidingly ramp against the ramp surface of the cage than the opposite sharp knife edge of the blade anchor which is configured to cut into vertebral bone for entry and anchoring therein. The anchors include sharp serrations 496 for assisting penetration into the bone space. The cross-section of a bone anchor may be T-shaped or triangular. The right anchor and left anchor are connected within the U-shaped right anchor-receiving location and left anchor-receiving location, respectively, by passing two pins through the pin apertures in the plate and the pin holes of the two anchors to attach the anchor to the plate to complete assembly of the anchor assembly. The pin-to-anchor attachment is shown to utilize two pins to attach the two anchors; however, the invention is not so limited and various attachment means are possible. The pin apertures in the plate and holes in the anchors are dimensioned with respect to the diameter of the pins such that a friction fit with frictional interference therebetween is sufficiently large so that the anchors do not flop around, rotate with respect to the plate or otherwise move under gravity or when being handled by the user. This feature advantageously maintains the anchors in an undeployed horizontal orientation so as to not interfere and remain in a low profile when manipulated by the user along the insertion pathway. Furthermore, the anchor 454 includes a first flat 497 and a second flat 498 both configured to abut the inner surface of the plate as can be seen in FIGS. 29A, 29B, 29D, 30C and 31D. The first flat 497 is a starting position flat as can be seen in FIG. 30C. The starting flat is a surface that abuts the plate and defines a first limit of rotation of the anchor. The second flat is a stop flat which prevents over rotation of the anchor with respect to the plate. The second flat is a surface that defines a second limit of rotation of the anchor. The anchor may rotate between the two limits. The second flat also defines and locks the final position of the bone anchor with respect to the plate. These flats on the anchor also appear on the previous variations of the spacer described above.

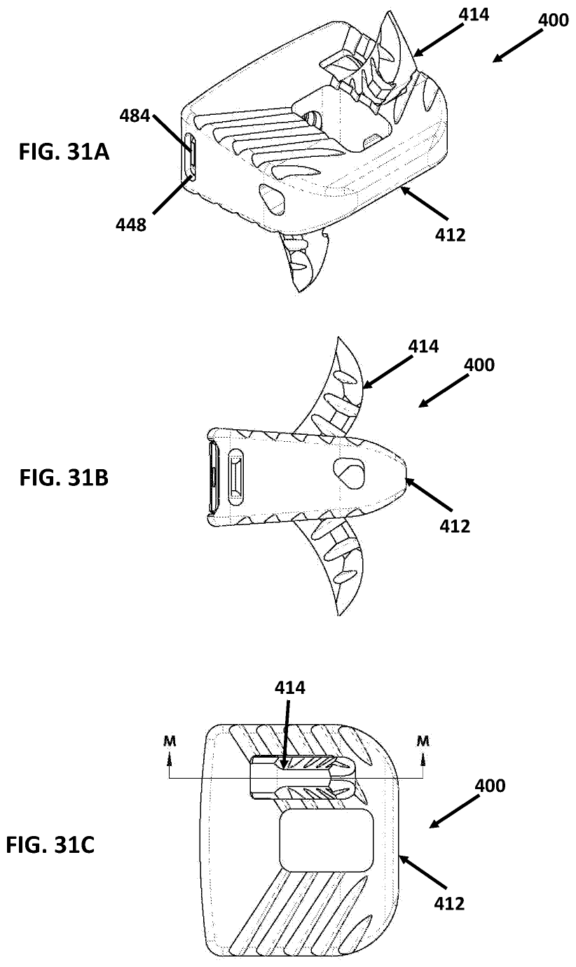

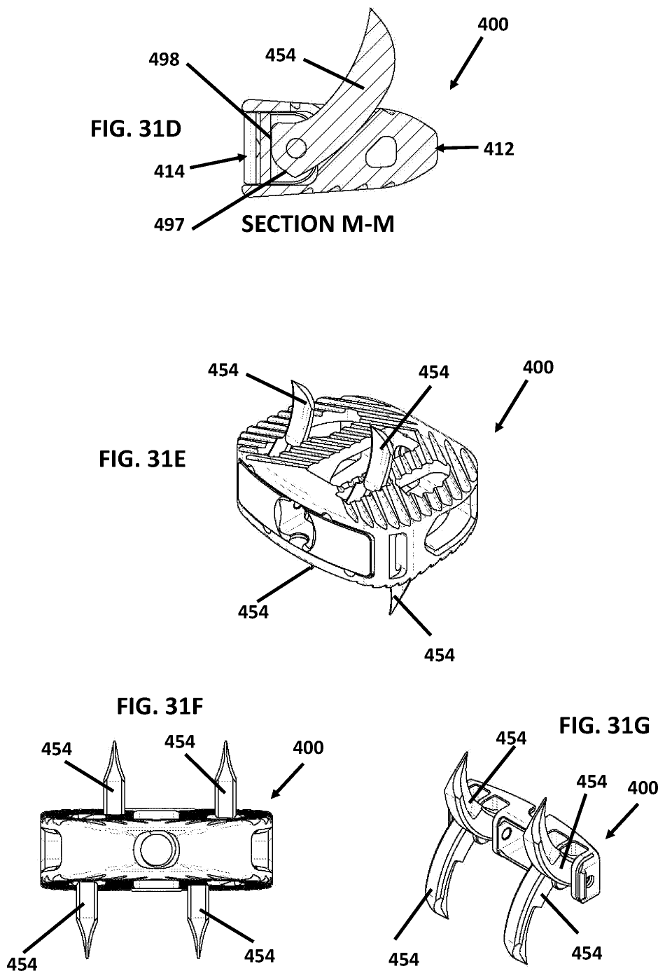

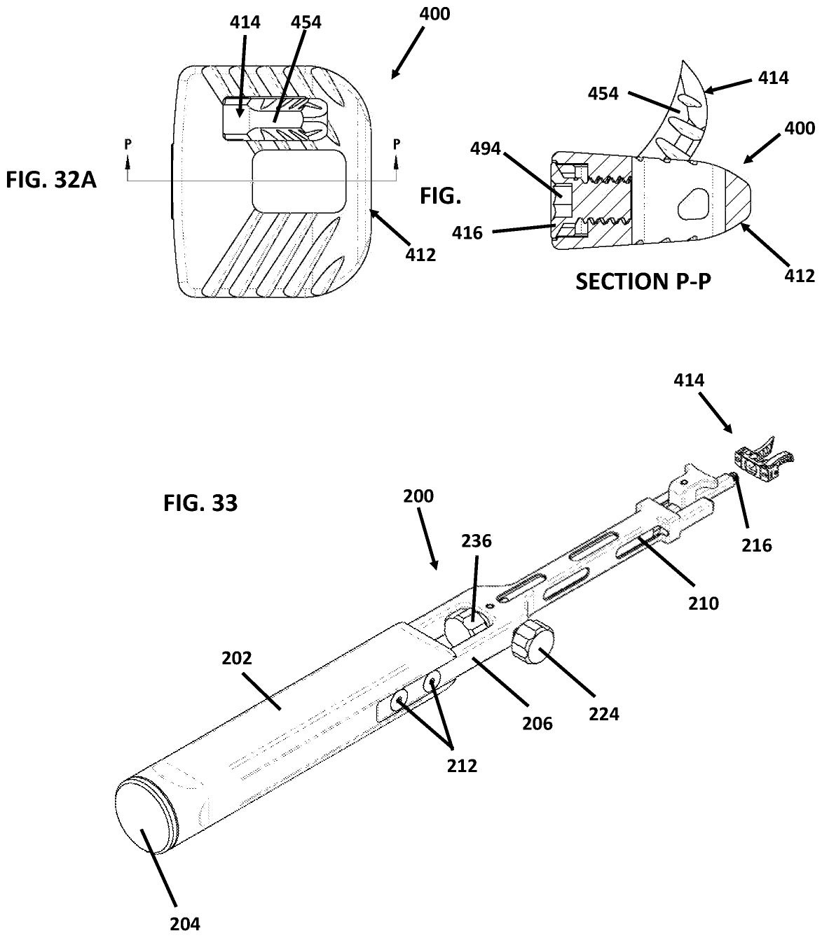

With particular reference to FIGS. 30A-30C and FIGS. 31A-31G, the above-described variation of the interbody spacer 400 comprises an anchor assembly 414 removably connectable to a cage 412. The anchor assembly includes two non-screw, bladed anchors—one anchor 454 configured to rotate up with respect to the plate and out of an exit opening of the cage for anchoring into an upper vertebral body and a second anchor configured to rotate down with respect to the plate and out of an exit opening of the cage for anchoring into a lower vertebral body. In an alternative variation, the spacer 400 is configured for four non-screw, bladed anchors—two anchors 454 configured to rotate up with respect to the plate and out of two exit openings in the cage for anchoring into an upper vertebral body and two bone anchors 454 configured to rotate down with respect to the plate and out of two exit openings in the cage for anchoring into the lower vertebral body as shown in FIGS. 31E-31G. In one variation, the four bone anchors have alternating rotations with a first anchor rotating up, a second anchor rotating down, a third anchor rotating up, and the fourth anchor rotating down. In another variation, the two middle anchors rotate up and the two outer anchors rotate down or vice versa. The cage includes a first anchor chamber 434 having a ramped surface for directing the rotation of one anchor upwardly and a second anchor chamber 438 having a ramped surface for directing the rotation of the second anchor downwardly. The anchor assembly is configured to be inserted anchor first in through the proximal opening 432 of the cage. Forward translation of the anchor assembly with respect to the cage for insertion of the anchor assembly into the cage causes the distal ends of the anchors to contact their respective ramped surface and, with continued forward translation, cause them overcome any friction with respect to the plate and to rotate with respect to the plate about their pins to exit such that one anchor projects into an upper vertebral body and the second anchor projects into a lower vertebral body when located within a disc space. Further distal translation of the anchor assembly causes the left and right locking tabs 478, 480 on either side of the plate to flex inwardly when the plate is inserted into the cage. The locking tabs have an angled/beveled distal tip that assists the insertion such that the angled tip ramps against the inner surface of the left and right sidewalls 422, 424 of the cage. The left locking tab 478 will deflect inwardly and then snap back outwardly when the left locking tab is no longer retained in a deflected state in the location of the left locking slot 450 into which the left hook will snap into. Similarly, the right locking tab 480 will deflect inwardly and then snap back outwardly when the right hook snaps into the right locking slot 448. The locking tabs 478, 480 have an angled distal end that aids insertion and deflection of the locking tabs 478, 480. The hooks 482, 484 have a vertical abutment at the proximal end that prevents the anchor assembly from backing out of the cage to lock the anchor assembly to the cage. The flexible locking tabs 478, 480 create a snap-fit engagement into the slots 448, 450 of the cage to lock the anchor assembly to the cage. The anchor assembly may be removed by employing an instrument inserted into the left and right locking slots to deflect the locking tabs inwardly to back the anchor assembly out of the cage. Alternatively, an instrument may be inserted into the apertures 485, 487 to deflect the locking tabs 478, 480 inwardly to release the hooks 482, 484 from the locking slots 448, 450. A secondary lock 416 depicted in FIG. 28 may be inserted into the instrument opening on the plate and threaded into the threaded bore 446 as shown in FIGS. 32A-32B. The secondary lock 416 is configured as a screw having a flat head 492 with a socket 494 as shown in FIG. 28. When the secondary lock 416 is inserted in through the instrument opening the distal threaded shaft will threadingly engage with the distal threaded bore 446 of the cage. The secondary lock is screwed to the cage capturing the locking assembly between the head 492 and the cage 412 to provide secondary backout protection for the anchor assembly 414 in addition to the primary backout protection provided by the snap lock action of the locking tabs 478, 480. This variation of the spacer describes backout protection designed with deflectable locking tabs on the plate that spring lock into slots on the cage.

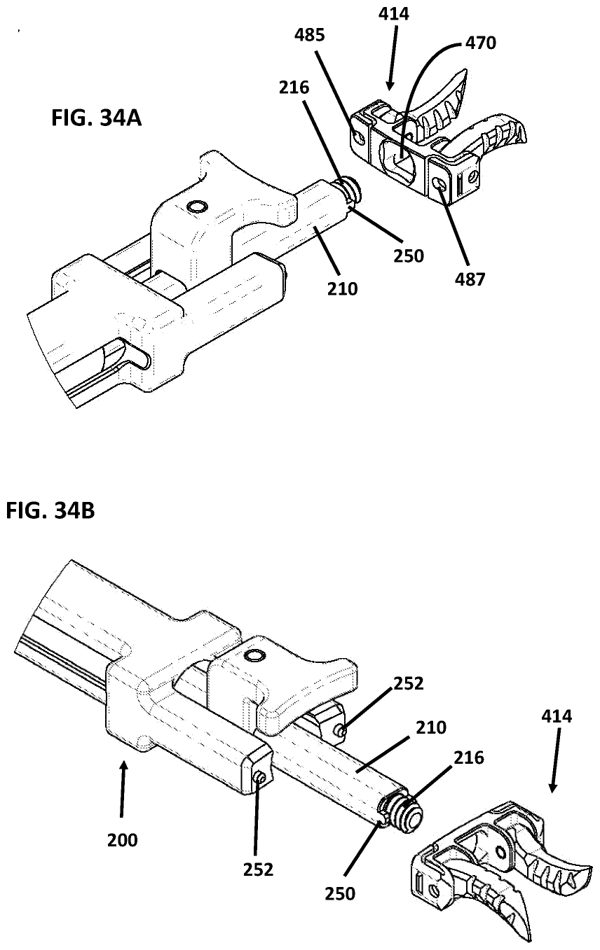

Turning now to FIGS. 33-36, the inserter instrument 200 is shown. The inserter instrument 200 is the same as described above with respect to FIGS. 20-22 with some minor differences configured for the interbody spacer 400. Continued reference is also made to FIGS. 20-22. The inserter 200 includes a handle 202 having a stainless-steel endcap 204 at the proximal end. A main body 206 is connected to the distal end. The main body has a forked proximal end and forked distal end. The central shaft 208 of the main body is rectangular in shape and has a central inner channel sized that is configured to receive a hollow sleeve 210 that is also rectangular in shape. The main body is connected to the handle by four flat head screws 212 to capture the sleeve and to capture an internal shaft 214 between the main body and handle such that the internal shaft can rotate relative to the handle, main body and sleeve. The internal shaft is provided with a proximal knob 236 for the user to rotate the internal shaft. The internal shaft has a threaded distal end 216 that is sized and configured to threadingly engage with the threaded bore 446 on the cage 412. The longitudinal translation of the sleeve is limited by a stop pin 218 inserted into the main body and into a slot 220 on the sleeve. The length of the slot on the sleeve serves as a proximal and distal abutment for the stop pin inserted therein to limit the longitudinal translation of the sleeve. Also, at the proximal end of the sleeve a hole 222 is provided that is sized and configured to receive the distal end of a lock 224. The lock is threaded into an aperture on the main body. There is a boss feature on the tip of the lock which goes into a hole on the sleeve which serves to lock the sleeve to the main body preventing the main body from moving relative to the sleeve. The distal end of the sleeve is provided with a depth stop pin hole 226 for receiving a depth stop pin 228. The depth stop pin is used to connect a U-shaped depth stop 230 to the sleeve. The depth stop has a distal surface 232 located on one side of the forked distal end of the main body so that the distal surface may contact against one of the upper or lower vertebral bodies to limit the depth of insertion of the spacer within the disc space. Furthermore, tangs 234 are not provided in the version of the inserter 200 modified for spacer 400. Instead, the forked distal end of the central shaft 208 includes a pair of posts 252 seen in FIG. 34B that are sized and configured for engagement and insertion into the apertures 485, 487 on the plate 452. The two posts 252 are located slightly inwardly closer to the midline longitudinal axis relative to the relaxed undeflected position of apertures 485, 487 such that the insertion of the posts 252 into the apertures 485, 487 slightly compresses the locking tabs 478, 480 to retain the bone anchor assembly to the inserter 200 via a friction fit. FIGS. 34A-34B illustrate the starting position of the inserter as it is positioned and correctly oriented with respect to the anchor assembly 414. The anchor assembly 414 is in an undeployed configuration in which the anchors 452 are in low-profile orientation extending along the longitudinal axis of insertion pathway. The starting position also includes the lock 224 being engaged into the sleeve. This means that the boss on the tip of the lock is in the hole of the sleeve. The user will know if this is the case if the main body does not slide relative to the sleeve. As can be seen in FIGS. 34A-34B, the distal end of the sleeve 210 is oriented and configured to fit inside the rear opening 432 of the cage. The distal end of the sleeve 210 includes two guides 250 located oppositely from each other and sized and configured for conforming engagement with the correspondingly shaped rectangular slots 431 formed in the cage 412 adjacent and through the threaded bore 446. As the distal end of the sleeve 210 is inserted into the rear opening 432, there is a first alignment made by the corresponding shape of the sleeve and the rear opening 432.

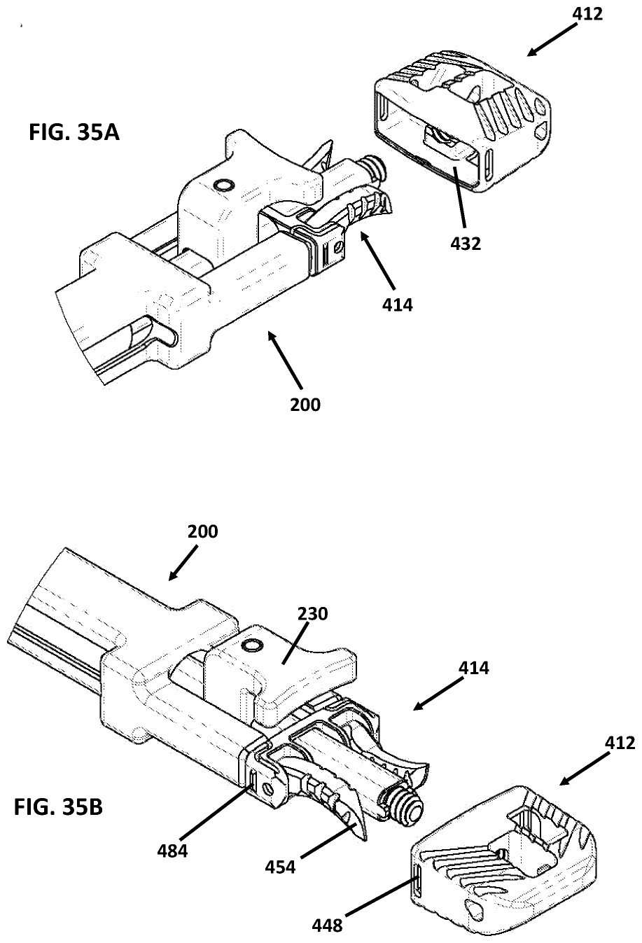

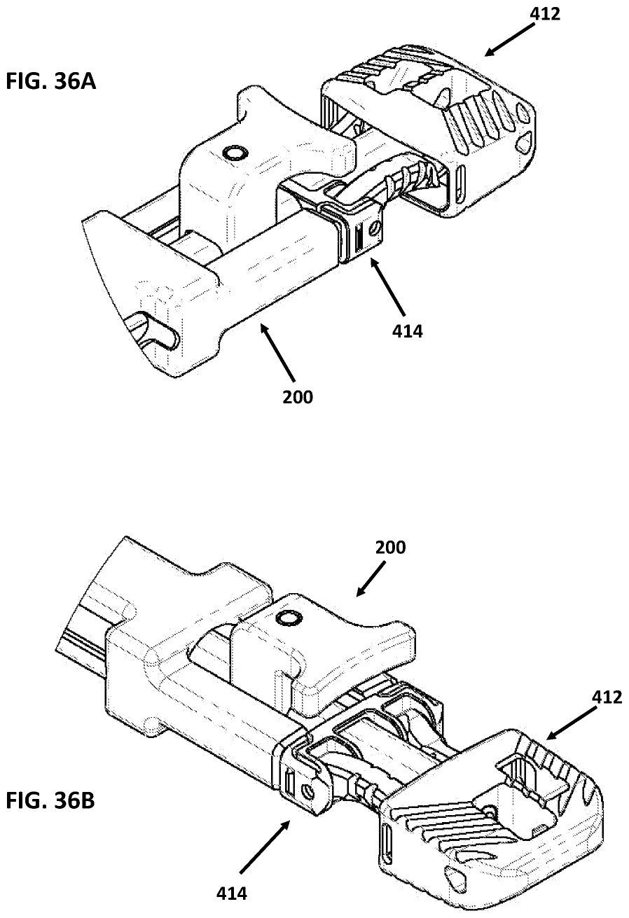

With the anchor assembly 414 loaded onto the distal end of the inserter instrument 200 via the friction fit between the posts 252 and apertures 485, 487, the instrument 200 is positioned as shown in FIGS. 35A-35B before the rear opening 432 of the cage 412 with the anchors 452 in a low-profile orientation. The anchor assembly 414 together with the instrument 200 is advanced into the cage 412 as shown in FIGS. 36A-36B. The threaded distal end 216 is threaded into the threaded bore 446 of the cage. As the distal end of the sleeve 210 is threaded into the threaded bore 446, the guides 250 will align cage with the respect to the rectangular slots 431. Lock 224 is activated to fix the main body 206 to the inner sleeve 210. This fixes the distance between the bone anchor assembly 414 and the cage 412 preventing the bone anchor assembly 414 from moving with relative to the cage so that the user can position the cage in the vertebral space. The user will then deliver the cage and anchor assembly connected to the instrument into the patient. With the cage in the desired position, the user unlocks the lock 224 to start the deployment of the bone anchor assembly 414 moving it with respect to the cage 412. As the anchor assembly 414 is advanced relative to the cage, the locking tabs 478, 480 will be deflected inwardly by left and right sidewalls 422, 424 of the cage 412 and will snap back outwardly such that the hooks 482, 484 are snapped and locked into the right and left locking slots 448, 450. Prior to completing this snap-locked position, the bone anchors 452 will have to undergo a force in order to penetrate into the upper and lower vertebrae. To accomplish this deployed configuration, the user may employ a mallet against the end cap 204 of the instrument 200 to move the anchor assembly and cage into position with respect to the vertebrae and to move the anchor assembly into position with respect to the cage. When the distal surface 232 of stop 230 abuts the vertebral bone, user will have an indication of final positioning of the cage in the disc space. The stop 230 is configured to be movable along the longitudinal axis of the instrument to adjust the position of the stop along the instrument. This allows the user to customize the insertion distance of the spacer into vertebral space.

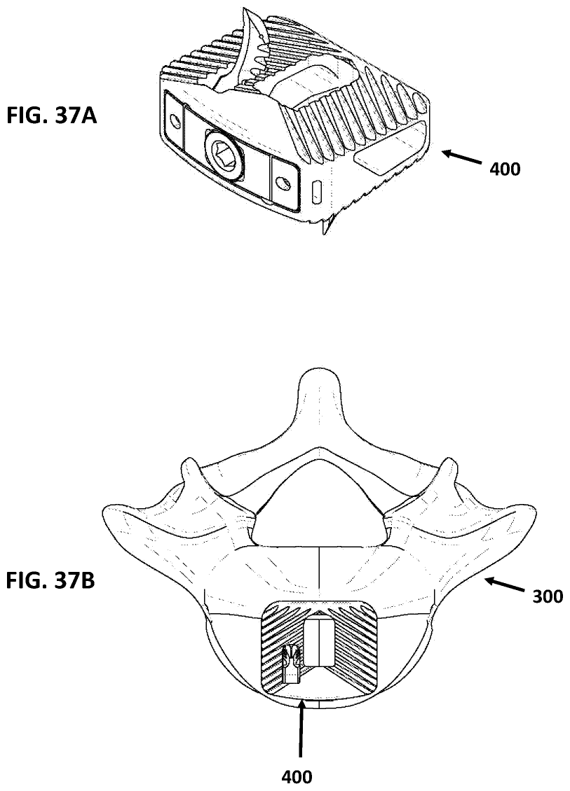

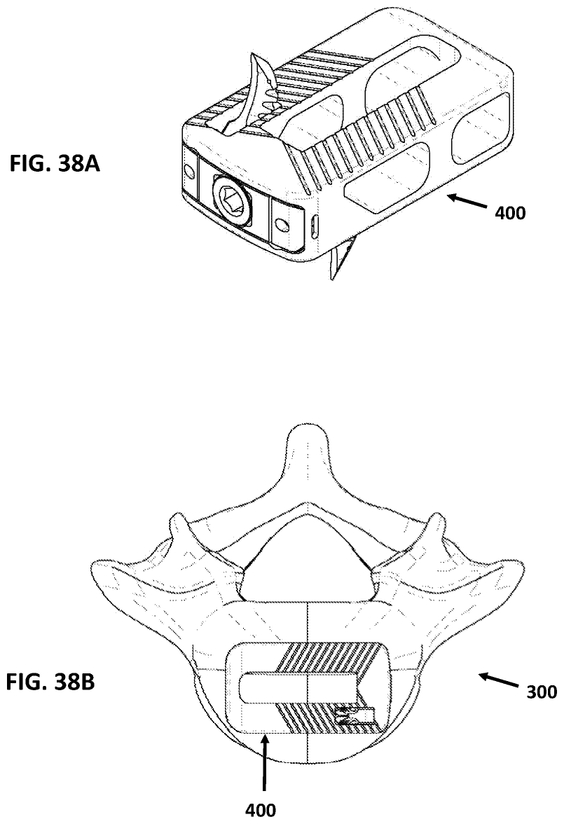

FIG. 37A there is shown an interbody spacer 400 shaped configured for anterior lumbar interbody fusion (ALIF) and its position in the disc space of a spinal column 300 is shown in FIG. 37B. FIG. 38A shows an interbody spacer configured for direct lateral interbody fusion (DLIF) according to the present invention and its position within a disc space of a spinal column 300 according to the present invention.

The interbody spacer of the present invention employs non-screw anchors. The non-screw anchors advantageously reduce the time to implant the spacer by reducing the time that it takes to screw a bone screw of a typical cage assembly. Furthermore, the anchors of the present invention are connected to the plate. As a result, delivery of the plate to the target space is advantageously simultaneous with delivery of the anchors; thereby, decreasing the surgical time and easing installation. Furthermore, the anchors are delivered simultaneously into both adjacent vertebral bodies; whereas conventional spacers require each anchor to be screwed into bone individually. The present invention deploys the anchors by hammering an inserter against the plate to which the anchors are connected. Taking advantage of the plate to distribute forces evenly prevents implant failure and allows for even and uniform deployment of the spacer into the vertebral space. The present invention advantageously employs ramps to rotate the anchors out of the exit ports in the cage eliminating any chance of torsional deformation. Furthermore, because the anchors are connected to the plate, the pullout strength is increased on the individual anchors compared to a system where the bone anchors are not connected. Furthermore, the entire spacer, cage, plate and anchors are preloaded onto the inserter which eliminates the need for multiple steps in delivery and the use of multiple instruments. The size of the spacer is extremely small, making it ideal for use in the cervical spine and can be sized for other locations along the spine as well. The spacer of the present invention further includes a unique anti-backout mechanism that employs dual action locking tabs to lock the anchor assembly to the cage. Furthermore, a secondary anti-backout lock is provided in the form of a screw which locks the anchor assembly to the cage for added protection.

It is understood that various modifications may be made to the embodiments of the mono endplate disclosed herein. Therefore, the above description should not be construed as limiting, but merely as exemplifications of preferred embodiments. Those skilled in the art will envision other modifications within the scope and spirit of the present disclosure.

I claim:

1. An interbody spacer comprising:
a cage including:
a top surface and a bottom surface interconnected by a left sidewall, a right sidewall and an endwall defining an interior having rear opening; a left locking slot formed in the left sidewall and a right locking slot formed in the right sidewall;
at least one ramped surface in the interior interconnected with a corresponding exit opening in the top surface;
at least one ramped surface in the interior interconnected with a corresponding exit opening in the bottom surface; and
a threaded bore in the interior facing the rear opening;
an anchor assembly including:
a plate configured for insertion into the rear opening and into the interior; the plate including an instrument opening aligned with the threaded bore; the plate including a left locking tab configured to flex with respect to the plate and enter into locking engagement with the left locking slot; the plate including a right locking tab configured to flex with respect to the plate and enter into locking engagement with the right locking slot; and
at least two non-screw bone anchors rotatably connected to the plate;
wherein the cage and anchor assembly are configured such that insertion of the anchor assembly into the cage causes the bone anchors to rotate out of the top and bottom exit openings and the left and right locking tabs to lock into the left and right locking slots.

2. The interbody spacer of claim 1 further including a secondary lock including a threaded shaft; the secondary lock sized to pass through the instrument opening and thread into the threaded bore to attach the plate to the cage.

3. The interbody spacer of claim 1 wherein the threaded bore is configured for threaded engagement with an insertion instrument.

4. The interbody spacer of claim 1 wherein the plate includes a left plate aperture and right plate aperture sized and configured to receive posts of an insertion instrument.

5. The interbody spacer of claim 1 wherein the plate includes a left hook extending outwardly from the left locking tab configured to snap into locking engagement with the left locking slot and a right hook extending outwardly from right locking tab configured to snap into locking engagement with the right locking slot.

6. The interbody spacer of claim 1 wherein an alignment slot is formed across the threaded bore for alignment with an insertion instrument.

7. An interbody spacer comprising:
a cage defining an interior and a rear opening;
an anchor assembly sized and configured for insertion into the rear opening and into the interior; the anchor assembly includes at least two non-screw bone anchors rotatably attached to an endplate.

8. The interbody spacer of claim 7 wherein the endplate is sized and configured to cover the rear opening.

9. The interbody spacer of claim 7 wherein the bone anchors are irremovably attached to the endplate.

10. The interbody spacer of claim 7 wherein insertion of the anchor assembly into the cage rotates the anchors into a deployed configuration.

11. The interbody spacer of claim 7 wherein the anchor assembly includes a low-profile undeployed configuration in which the bone anchors are oriented for insertion into the cage and a high-profile deployed configuration in which the anchor assembly is inside the cage and the bone anchors extend out of the cage.

12. The interbody spacer of claim 7 wherein insertion of the anchor assembly into the cage locks the endplate to the cage.

13. The interbody spacer of claim 7 wherein the cage includes deflectable locking tabs sized and configured for locking engagement with the endplate.

14. The interbody spacer of claim 7 wherein the endplate includes deflectable locking tabs sized and configured for locking engagement with the cage.

15. The interbody spacer of claim 7 wherein each anchor includes a first flat surface configured to abut an inner surface of the endplate and a second flat surface configured to abut the inner surface of the endplate to define two limits of rotation for each anchor.

16. The interbody spacer of claim 7 wherein the anchor assembly includes one bone anchor configured to deploy through the top of the cage and one bone anchor configured to deploy through the bottom of the cage.

17. The interbody spacer of claim 7 wherein the anchor assembly includes two bone anchors configured to deploy through the top of the cage and two bone anchors configured to deploy through the bottom of the cage.

18. An interbody spacer system comprising:
an interbody spacer including a cage and an anchor assembly; the anchor assembly including at least two non-screw bone anchors rotatably connected to a plate; the plate having an instrument opening having a cross-sectional shape; and
an insertion instrument including a main body having a cross-sectional shape corresponding to the shape of the instrument opening and sized and configured for insertion into the instrument opening.

19. The interbody spacer system of claim 18 wherein the cross-sectional shape is non-circular.

20. The interbody spacer system of claim 18 wherein the insertion instrument further includes a lock to fix the distance between the cage and anchor assembly for insertion of the spacer.
